SEQUENCE LISTING (21 sequences)

SEQ ID NO: 1 - DNA, 23 bp, Zea mays
  source 1..23: mol_type = genomic DNA
tttcgtccagtaaaattaattaa

SEQ ID NO: 2 - DNA, 23 bp, Zea mays
  source 1..23: mol_type = genomic DNA
gtccagtaaaattaattaaggtg

SEQ ID NO: 3 - DNA, 23 bp, Zea mays
  source 1..23: mol_type = genomic DNA
ctggacgaaataaatcactagta

SEQ ID NO: 4 - DNA, 23 bp, Zea mays
  source 1..23: mol_type = genomic DNA
cagccttgtgctagccgcagcca

SEQ ID NO: 5 - DNA, 23 bp, Zea mays
  source 1..23: mol_type = genomic DNA
ttctagagaaatattatatcgtg

SEQ ID NO: 6 - DNA, 23 bp, Zea mays
  source 1..23: mol_type = genomic DNA
ttgtccactatccatccagtggg

SEQ ID NO: 7 - DNA, 23 bp, Zea mays
  source 1..23: mol_type = genomic DNA
cgagacggagcactatattattg

SEQ ID NO: 8 - DNA, 23 bp, Zea mays
  source 1..23: mol_type = genomic DNA
tgtatccgcaaaacggcagccgt

SEQ ID NO: 9 - DNA, 23 bp, Zea mays
  source 1..23: mol_type = genomic DNA
cttgccgccctcgtggttggaat

SEQ ID NO: 10 - DNA, 23 bp, Zea mays
  source 1..23: mol_type = genomic DNA
ccaaactccattcagccattcgc

SEQ ID NO: 11 - DNA, 23 bp, Zea mays
  source 1..23: mol_type = genomic DNA
gtgtctagtgtgtgcgaagatgg

SEQ ID NO: 12 - DNA, 23 bp, Zea mays
  source 1..23: mol_type = genomic DNA
tacgccatcttcgcacacactag

SEQ ID NO: 13 - DNA, 23 bp, Zea mays
  source 1..23: mol_type = genomic DNA
ttgtatgcgctgatctgattctg

SEQ ID NO: 14 - DNA, 23 bp, Zea mays
  source 1..23: mol_type = genomic DNA
cggcagcgcgtcgtagcagttct

SEQ ID NO: 15 - DNA, 23 bp, Zea mays
  source 1..23: mol_type = genomic DNA
cttaattttctttcttcatatca

SEQ ID NO: 16 - DNA, 23 bp, Zea mays
  source 1..23: mol_type = genomic DNA
tttcttcatatcatgcactctgg

SEQ ID NO: 17 - DNA, 23 bp, Zea mays
  source 1..23: mol_type = genomic DNA
ttcatatcatgcactctggctgg

SEQ ID NO: 18 - DNA, 23 bp, Zea mays
  source 1..23: mol_type = genomic DNA
gaatccacgacatgcaagagctc

SEQ ID NO: 19 - DNA, 23 bp, Zea mays
  source 1..23: mol_type = genomic DNA
attgctctcttttaatttggtga

SEQ ID NO: 20 - DNA, 23 bp, Zea mays
  source 1..23: mol_type = genomic DNA
atttggtgaagaatcgaatcgat

SEQ ID NO: 21 - DNA, 23 bp, Zea mays
  source 1..23: mol_type = genomic DNA
gtgaagaatcgaatcgattctac

(19) United States
(12) Patent Application Publication
(10) Pub. No.: US 2023/0091138 A1
(43) Pub. Date: Mar. 23, 2023

(54) METHODS AND SYSTEMS FOR USE IN IDENTIFYING GUIDE NUCLEIC ACID SEQUENCES CONSISTENT WITH EXPERIMENTAL SCALING

(71) Applicant: MONSANTO TECHNOLOGY LLC, Saint Louis, MO (US)

(72) Inventors: Thomas REAM, Wildwood, MO (US); Linda RYMARQUIS, High Ridge, MO (US)

(21) Appl. No.: 17/817,551

(22) Filed: Aug. 4, 2022

Related U.S. Application Data
(60) Provisional application No. 63/230,025, filed on Aug. 5, 2021.

(51) Int. Cl. G16B 40/20; G16B 30/10; G16B 20/00

(52) U.S. Cl. CPC G16B 40/20; G16B 30/10; G16B 20/00

(57) ABSTRACT

Systems and methods for identifying mechanisms for editing genome sequences are provided. One example computer-implemented method includes, for each of multiple guide nucleic acid sequences, for a desired edit: identifying characteristics of the guide nucleic acid sequence and/or sequence segment; assigning, based on a scoring data structure, a score to the guide nucleic acid sequence for each identified characteristic; and aggregating the assigned scores into an edit score for the guide nucleic acid sequence. The method then includes compiling a report that includes the multiple guide nucleic acid sequences and the edit score for each of the guide nucleic acid sequences, thereby permitting selection, from the report, of at least one of the guide nucleic acid sequences based on the associated edit score. Additionally, based on the edit score, a number of guide nucleic acid sequences tested, a sample size, and/or a number of experiments can be set to reach the desired edit.

### CROSS-REFERENCE TO RELATED APPLICATION

This application claims the benefit of, and priority to, U.S. Provisional Application No. 63/230,025, filed Aug. 5, 2021, the entire contents of which is hereby incorporated by reference.

### FIELD

The present disclosure generally relates to methods and systems for use in selecting, identifying, using, etc. guide nucleic acid sequences (e.g., gRNA sequences (or, generally, gRNAs), gRNAs/DNAs, gDNAs, etc.) and, in particular, to methods and systems for identifying guide nucleic acid sequences consistent with sample and/or experimental scaling to promote gene editing.

### INCORPORATION OF SEQUENCE LISTING

A sequence listing contained in the file named “BCS216106US01_ST26_1” which is 19,268 bytes (measured in MS-Windows®) and created on Aug. 3, 2022, is filed electronically herewith and incorporated by reference in its entirety

### BACKGROUND

This section provides background information related to the present disclosure which is not necessarily prior art.

In plant breeding, modifications are made in plants, either through crossing and selection of plants with desirable traits (“conventional breeding”) or through direct genetic manipulation (e.g., use of transgenes, gene editing, etc.). Conventional breeding techniques for improving plant stocks is resource intensive, requiring many hundreds, thousands, or more crosses (depending on the number of traits to be introgressed into a given stock) over multiple generations and is limited by the alleles pre-existing in the population. Genome editing technologies, in particular the clustered regularly interspersed short palindromic repeats (CRISPR) technology, can effectuate very precise modifications of the genome, improving genetic diversity and accelerating the process of introducing traits into a germline by reducing the number of cross-matings necessary to generate a stable line with the desired traits. The process of transforming the CRISPR technology into plant cells and screening the primary transformants to identify edits is resource intensive. Methods and systems that increase the frequency of obtaining desired edits or allow for scaling the number of primary transformants to ensure edits are recovered without wasting resources, is highly desirable.

### SUMMARY

This section provides a general summary of the disclosure, and is not a comprehensive disclosure of its full scope or all of its features.

Example embodiments of the present disclosure generally relate to methods for identifying one or more guide nucleic acids (e.g., gRNAs, gRNAs/DNAs, gDNAs, etc.) (broadly, mechanisms) for editing a genome sequence.

In one example embodiment, a method includes, for each of multiple guide nucleic acid (e.g., gRNA, gRNA/DNA, gDNA, etc.) sequences, for a desired edit of a sequence segment of a target organism: (i) identifying, by a genome editor computing device, one or more characteristics of the guide nucleic acid sequence; (ii) assigning, by the genome editor computing device, based on a scoring data structure, a score to the guide nucleic acid sequence for each of the identified one or more characteristics; and (iii) aggregating, by the genome editor computing device, the assigned scores into an edit score for the guide nucleic acid sequence. The method then also includes compiling, by the genome editor computing device, a report, wherein the report includes the multiple guide nucleic acid sequences and the edit score for each of the multiple guide nucleic acid sequences, thereby permitting selection, from the report, of at least one of the multiple guide nucleic acid sequences based on the associated edit score.

In addition, the method of this example embodiment may also include identifying the at least one of the multiple guide nucleic acid sequences, from the report, based on the associated edit score, and/or determining, by the genome editor computing device, a number of experiments and/or samples for the at least one of the multiple guide nucleic acid sequences, based on the edit score and a defined effectivity rate from the request and/or based on an effectivity rate of the at least one of the multiple guide nucleic acid sequences.

In another example embodiment, a method includes, for each of multiple guide RNA (gRNA) sequences, for a desired edit of a sequence segment of a target organism: (i) identifying, by a genome editor computing device, one or more characteristics of the gRNA sequence; (ii) assigning, by the genome editor computing device, based on a scoring data structure, a score to the gRNA sequence for each of the identified one or more characteristics; and (iii) aggregating, by the genome editor computing device, the assigned scores into an edit score for the gRNA sequence. The method then includes determining, by the genome editor computing device, for at least one of the multiple gRNA sequences, a number of experiments and/or samples to achieve the desired edit of the sequence segment of the target organism, based on the edit score for the at least one of the multiple gRNA sequences.

In addition in this example embodiment, determining the number of experiments and/or samples for the at least one of the multiple guide nucleic acid sequences to achieve the desired edit of the sequence segment of the target organism may further be based on an effectivity rate of the at least one of the multiple guide nucleic acid sequences and/or a defined effectivity rate included in a request for the desired edit.

Example embodiments of the present disclosure also generally relate to systems for use in identifying one or more guide nucleic acids (e.g., gRNAs, gRNAs/DNAs, gDNAs, etc.) (broadly, mechanisms) for editing a genome sequence.

In one example embodiment, a system includes a genome editor computing device configured to, for each of multiple guide nucleic acid (e.g., gRNA, gRNA/DNA, gDNA, etc.) sequences, for a desired edit of a sequence segment of a target organism: (i) identify one or more characteristics of the guide nucleic acid sequence; (ii) assign, based on a scoring data structure, a score to the guide nucleic acid sequence for each of the identified one or more characteristics; and (iii) aggregate the assigned scores into an edit score for the guide nucleic acid sequence. The genome editor computing device is then further configured to store, in memory in communication with the genome editor computing device, the multiple guide nucleic acid sequences and the edit score for each of the multiple guide nucleic acid sequences, thereby permitting selection of at least one of the multiple guide nucleic acid sequences based on the associated edit score.

In addition in this example embodiment, the genome editor computing device may be further configured to determine a number of experiments and/or samples for the at least one of the multiple guide nucleic acid sequences, based on the edit score and a defined effectivity rate from the request and/or based on an effectivity rate of the at least one of the multiple guide nucleic acid sequences.

In another example embodiment, a system includes at least one genome editor computing device configured to receive a request for a desired edit of a sequence segment of a target organism and identify multiple gRNA sequences based on the desired edit of the sequence segment of the target organism and/or based on a location in the sequence segment in the target organism. The at least one genome editor computing device is further configured, for each of the identified multiple guide RNA (gRNA) sequences, for the desired edit, to: (i) identify one or more characteristics of the gRNA sequence; (ii) assign, based on a scoring data structure, a score to the gRNA sequence for each of the identified one or more characteristics; and (iii) aggregate the assigned scores into an edit score for the gRNA sequence. And, the at least one genome editor computing device is then configured to compile a report, wherein the report includes the multiple gRNA sequences and the edit score for each of the multiple gRNA sequences, thereby permitting selection, from the report, of at least one of the multiple gRNA sequences based on the associated edit score.

In addition in this example embodiment, the genome editor computing device may be further configured to determine a number of experiments and/or samples for the at least one of the multiple guide nucleic acid sequences, based on the edit score and a defined effectivity rate from the request and/or based on an effectivity rate of the at least one of the multiple guide nucleic acid sequences.

In another example embodiment, a system includes at least one genome editor computing device configured, for each of multiple guide RNA (gRNA) sequences, for a desired edit of a sequence segment of a target organism, to: (i) identify one or more characteristics of the gRNA sequence; (ii) assign, based on a scoring data structure, a score to the gRNA sequence for each of the identified one or more characteristics; and (iii) aggregate the assigned scores into an edit score for the gRNA sequence. The at least one genome editor computing device is then further configured to determine, for at least one of the multiple gRNA sequences, a number of experiments and/or samples to achieve the desired edit of the sequence segment of the target organism, based on the edit score for the at least one of the multiple gRNA sequences.

In addition in this example embodiment, the genome editor computing device may be configured, in order to determine the number of experiments and/or samples for the at least one of the multiple guide nucleic acid sequences to achieve the desired edit of the sequence segment of the target organism, to determine the number of experiments and/or samples further based on an effectivity rate of the at least one of the multiple guide nucleic acid sequences and/or a defined effectivity rate included in a request for the desired edit.

Example embodiments of the present disclosure also generally relate to non-transitory computer-readable storage media including executable instructions for identifying one or more guide nucleic acids (e.g., gRNAs, gRNAs/DNAs, gDNAs, etc.) (broadly, mechanisms) for editing a genome sequence, which when executed by at least one processor of a genome editor computing device, cause the at least one processor to perform one or more of the operations recited above.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, are not all possible implementations, and are not intended to limit the scope of the present disclosure.

FIG. 1 illustrates an example system of the present disclosure configured for identifying suitable gRNAs for use in editing genome projects, based on scores associated with the suitable gRNAs;

FIG. 2 illustrates an example graphical representation of a rate of effectivity of a gRNA based on a score associated with the gRNA;

FIG. 3 is a block diagram of a computing device that may be used in the example system of FIG. 1;

FIG. 4 is an example method, suitable for use with the system of FIG. 1, for identifying a suitable gRNA for use in editing a genome sequence, based on an effectivity rate of a score associated with the suitable gRNA;

FIGS. 5A and 5B illustrate example graphical representations of a number of gRNAs having rates of effectivity as defined before (5A) and after (5B) the scoring described herein; and

FIG. 6 illustrates an example report that may be compiled in connection with operation of the system of FIG. 1 and/or application of the method of FIG. 4.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Genome editing techniques, such the use of CRISPR systems, etc., may be implemented to modify genomic sequences. These modifications can include edits by, for example, introducing breaks at one or more targeted locations in the genome, which when repaired, can result in simple or complex edits, including, for example, deletions, insertions, translocations and inversions. Other examples of modifications can include edits introduced by hydrolytic deamination of cytidine or deoxycytidine to uridine or deoxyuridine, respectively. Genome editing techniques can also be used to modify genomes through epigenetic modifications resulting in gene activation or repression. Use of genome editing techniques can enhance processes associated with introducing traits into genomes and, potentially, allow for the stacking of linked traits whose genomic loci are in repulsion. With genome editing techniques, it is intended, generally, to introduce modifications, such as nucleic acid breaks or hydrolytic deamination, at particular locations to facilitate the edits, where the effectiveness of the techniques is often associated with effectivity rates, which define the effectivity (or effectiveness) of each of the techniques as a percentage. For a particular guide nucleic acid (e.g., gRNA, gRNA/DNA, gDNA, etc.), for example, an effectivity rate of 30 percentage may be defined (e.g., through experimentation, etc.). That said, the effectivity rates may vary based on the specific guide nucleic acids, the characteristics of the sequence segment, or the complexity of the edits to be made, and the plants or organisms, more generally, to which the edits are directed.

Uniquely, the methods and systems herein enable the selection of a guide nucleic acid to perform a desired edit, in response to a score, for the guide nucleic acid, having a specific effectivity rate satisfying a desired threshold, for example, as compared to other guide nucleic acids.

In particular herein, a researcher, breeder or other user requests an edited sequence, and in doing so, defines, among other things, an organism (e.g., plant, etc.), an input sequence for the organism, the CRISPR effector protein (e.g., Cas9, Cas12a, etc.) and a specific edit and/or an editing region in the input sequence, etc. to an editor computing device. In some embodiments, the input sequence for the organism can be within the reference genome for a species of the said organism. In response, the editor computing device identifies each available guide nucleic acid for the edit of the input sequence, and then scores the available guide nucleic acids, for example, based on the guide nucleic acids and taking into account different characteristics of the target organism, CRISPR editing system, guide nucleic acids and/or the sequence segment. The editor computing device further outputs the identified guide nucleic acid(s) and associated score(s) to the researcher, breeder or other user. In connection therewith, based on an effectivity rate defined by the researcher, breeder or other user, the editor computing device may also, optionally, select a specific one or more guide nucleic acid(s) based on the scores and/or a number of samples/experiments to be executed in order to satisfy the request by the researcher, breeder or other user for the edit(s) to the input sequence. In some embodiments, based on an effectivity rate of a selected guide nucleic acid defined by the researcher, breeder or other user, the editor computing device may also, optionally, select a number of samples to be edited with a CRISPR system comprising the selected guide nucleic acid in order to satisfy the request by the researcher, breeder or other user to recover edit(s) to the input sequence. In this manner, an objective measure of the estimated effectivity rates for different identified guide nucleic acids, for different organisms and edits, etc., is provided, whereby a researcher, breeder or other user (or the editor computing device) may select appropriate guide nucleic acids (at the location, as based on score, etc.) and/or number of samples to be edited to provide sufficient confidence in recovering the desired output sequence and/or to conserve resources associated with the editing of the input sequence, etc.

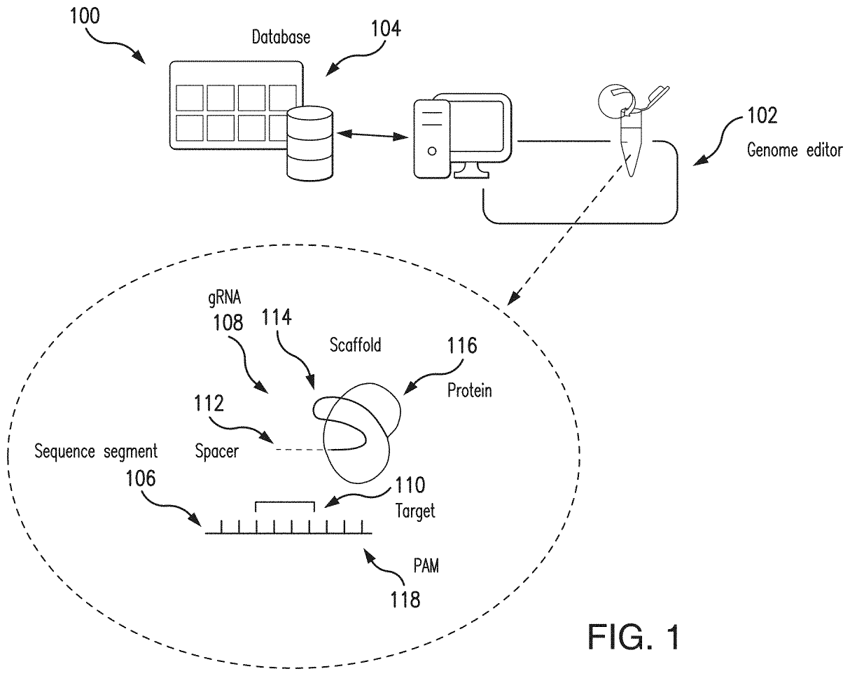

FIG. 1 illustrates an example system 100 in which one or more aspects of the present disclosure may be implemented. Although the system 100 is presented in one arrangement, other embodiments may include the parts of the system 100 (or additional parts) arranged otherwise depending on, for example, the manner in which genome edits are identified, selected, and/or edited into a sequence of an organism, the types and/or number of genome edits, etc.

In the example embodiment of FIG. 1, the system 100 generally includes an editor 102 (e.g., a genome editor, an editor computing device, a genome editor computing device, etc.) and a database 104 coupled in communication with the editor 102 via one or more network connections such as, for example, one or more of a local area network (LAN), a wide area network (WAN) (e.g., the Internet, etc.), a mobile network, a virtual network, and/or another suitable public and/or private network capable of supporting wired and/or wireless communication therebetween. As such, the editor 102, more generally, is accessible directly by a researcher, breeder or other user (e.g., at the editor computing device, etc.), or potentially, via the one or more network connections (e.g., as a web-based program or service, etc.).

As will be described, the editor 102 is programmed or configured to identify one or more gRNAs for an input sequence (e.g., a genome sequence, an input sequence segment, a target sequence, etc.) of a target organism, and to assess potential success of each gRNA for making desired edits to the input sequence by providing a score therefor. The edits may include various types of edits to the input sequence, including, without limitation, insertion edits, deletion edits, inversion edits, substitution edits, translocation edits, gene activation/repression, epigenetic modifications, hydrolytic deaminations or combinations thereof (e.g., by way of, through use of, through application of, etc. one or more editing technologies; etc.). The database 104 is programmed or configured to include various data structures, as described below, data from which is used by the editor 102 to evaluate guide RNAs (gRNAs) for making the desired edits.

In general, the system 100 employs CRISPR technology (e.g., CRISPR/Cas technology such as, for example, a Type I CRISPR-Cas system, a Type II CRISPR-Cas system (e.g., a Cas9 system, etc.), a Type III CRISPR-Cas system, a Type IV CRISPR-Cas system, a Type V CRISPR-Cas system (e.g., a Cas12a (Cpf1) system, a Cas12b system, a Cas12c (C2c3) system, a Cas12d (CasY) system, a Cas12e (CasX) system, a Cas12g system, a Cas12h system, a Cas12i system, a C2c1 system, a C2c4 system, a C2c5 system, a C2c8 system, a C2c9 system, a C2c10 system, a Cas14a system, a Cas14b system, a Cas14c system, etc.), a Type VI CRISPR-Cas system, etc.) to achieve the editing described herein. In this example embodiment, the CRISPR-Cas system (the protein of 116 complexed with the guide nucleic acid of 108) is provided to aid in precise genomic editing of the input sequence (which includes at least a part of the genomic sequence of the target organism). The technology permits a breeder, researcher or other user to choose a precise location within the input sequence to edit by engineering a guide nucleic acid (e.g., gRNA) 108 that will match a target 110 of the input sequence at a desired location proximal to a protospacer-adjacent motif (PAM) sequence 118. As shown in FIG. 1, for example, the guide nucleic acid (e.g., gRNA) 108 (also termed a gRNA sequence) includes a spacer sequence 112 and a scaffold sequence 114. The spacer sequence 112 is configured to bind to the specific target 110 in the input sequence, where editing is desired. The scaffold sequence 114 is configured for binding to a CRISPR effector protein 116, in this example where, consistent with the above, the CRISPR effector protein 116 includes unmodified CRISPR effector proteins, dead CRISPR effector proteins, nickase CRISPR effector proteins, CRISPR effector fusion proteins (e.g., deaminase, reverse transcriptase, transposase, glycosylase, glycosylase inhibitor, methylase, demethylase, methyltransferase, helicase, ligase, polymerase, etc. CRISPR effector fusion proteins), peptide tagged CRISPR effector proteins, etc.

In some embodiments, the CRISPR technology may be a Type II Clustered Regularly Interspaced Short Palindromic Repeats (CRISPR) Cas9 system. Cas9 recognizes a G-rich protospacer-adjacent motif (PAM) that is 3′ to its target site (protospacer, guide binding site, target nucleic acid, target DNA) (3′-NGG). Cas9 effector proteins' nuclease activity produces blunt double stranded breaks. In other embodiments, the CRISPR technology may be a Type V CRISPR Cas12a system. Cas12a recognizes a T-rich PAM that is located 5′ to the target nucleic acid (5′-TTN, 5′-TTTN). Cas12a effector proteins' nuclease activity produces staggered DNA double stranded breaks.

A “guide nucleic acid,” “guide RNA (gRNA)”, “guide RNA/DNA (g(RNA/DNA)”, or “guide DNA (gDNA)” as used herein means a nucleic acid that complexes with a CRISPR effector protein and guides the CRISPR system (the CRISPR effector protein complexed to the guide nucleic acid) to the target. A guide nucleic acid comprises a “spacer sequence” or “spacer” or “crRNA”, which is complementary to (and hybridizes to) a target sequence, and a “repeat sequence” or “scaffold sequence” or “scaffold” or “tracrRNA” that interacts with (binds to) the CRISPR effector protein. A guide nucleic acid may be a single nucleic acid molecule (e.g., sgRNA) or two separate nucleic acid molecules (e.g., a 2-piece gRNA). A guide nucleic acid can be configured such that the repeat sequence/scaffold is linked to the 5′ end and/or the 3′ end of the spacer sequence. In some embodiments, the guide nucleic acid comprises DNA. In some embodiments, the guide nucleic acid comprises RNA. In some embodiments, the guide nucleic acid comprises both DNA and RNA. The design of a guide nucleic acid may be based on a Type I, Type II, Type III, Type IV, Type V, or Type VI CRISPR-Cas system. In some embodiments, a guide nucleic acid may comprise, from 5′ to 3′, a scaffold sequence and a spacer sequence. In some embodiments, a guide nucleic acid may comprise, from 5′ to 3′, a spacer sequence and a scaffold sequence. In some embodiments, a guide nucleic acid may comprise more than one scaffold sequence-spacer sequence (e.g., scaffold-spacer-scaffold, scaffold-spacer-scaffold-spacer-scaffold-spacer, etc.). In some embodiments, a guide nucleic acid may comprise 2, 3, 4, 5, 6, 7, 8, 9, 10, or more spacer-scaffold sequences. The spacer sequences may be configured to bind to the same or different targets. In some embodiments, there may be two or more (e.g., 2, 3, 4, 5, or more) different targets and two more (e.g., 2, 3, 4, 5, or more) different spacers. In some embodiments a guide nucleic acid may further comprise an RNA template (pegRNA) for a reverse transcriptase.

In some embodiments, the spacer sequence is 100% complementary to a target sequence. In other embodiments, a spacer sequence is substantially complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) to a target sequence. In some embodiments, a spacer sequence can have one, two, three, four, or five contiguous or noncontiguous mismatches as compared to the target sequence. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). In some embodiments, a spacer sequence may have complete complementarity or substantial complementarity over a region of a target sequence that is at least about 15 nucleotides to about 30 nucleotides in length. In some embodiments, the 5′ region of a spacer sequence of a guide nucleic acid may be fully complementary to a target sequence, while the 3′ region of the spacer may be substantially complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) to the target sequence. In other embodiments, the 5′ region of a spacer sequence of a guide nucleic acid may be substantially complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) to a target sequence, while the 3′ region of the spacer may be fully complementary to the target sequence. In some embodiments, the first 1, 2, 3, 4, 5, 6, 7, 8, 9, 10, 11, 12, 13, 14, 15 or more nucleotides in the 5′ region of a spacer sequence may be 100% complementary to the target, while the remaining nucleotides in the 3′ region of the spacer sequence are substantially complementary to the target. In some embodiments, the first 1, 2, 3, 4, 5, 6, 7, 8, 9, 10, 11, 12, 13, 14, 15 or more nucleotides in the 5′ region of a spacer sequence may be substantially complementary to the target, while the remaining nucleotides in the 3′ region of the spacer sequence are 100% complementary to the target.

A “repeat sequence” or “scaffold sequence” or “scaffold” or “tracrRNA” can be any repeat sequence of any known or later identified CRISPR Cas locus (e.g., a Type I, Type II, Type III, Type IV, Type V or Type VI locus, a Cas9 locus, a Cas12a locus, a C2c1 locus, etc.) or a synthetic repeat sequence (scaffold) designed to function with the selected CRISPR-Cas effector protein. A scaffold sequence may comprise a hairpin structure and/or a stem loop structure. In some embodiments, a scaffold sequence may form a pseudoknot-like structure at its 5′ end. In some embodiments, a scaffold sequence comprises about 10 to about 15, about 10 to about 20, about 10 to about 30, about 10 to about 45, about 10 to about 50 or more nucleotides.

While the spacer sequence 112 and the scaffold sequence 114 of the gRNA 108 are illustrated as engineered together in this embodiment, it should be appreciated that the gRNA 108 may be engineered with the scaffold sequence 114 and the spacer sequence 112 separately or in partial combinations. In all cases, the region corresponding to the spacer sequence 112 is scored. In some embodiments, scoring metrics may be applied to both the spacer sequence 112 and all or part of the scaffold sequence 114 together.

What's more in the system 100, the CRISPR effector protein 116 (e.g., a Type I CRISPR-Cas effector protein, a Type II CRISPR-Cas effector protein (e.g., a Cas9 effector protein, etc.), a Type III CRISPR-Cas effector protein, a Type IV CRISPR-Cas effector protein, a Type V CRISPR-Cas effector protein (e.g., a Cas12a (Cpf1) effector protein, a Cas12b effector protein, a Cas12c (C2c3) effector protein, a Cas12d (CasY) effector protein, a Cas12e (CasX) effector protein, a Cas12g effector protein, a Cas12h effector protein, a Cas12i effector protein, a C2c1 effector protein, a C2c4 effector protein, a C2c5 effector protein, a C2c8 effector protein, a C2c9 effector protein, a C2c10 effector protein, a Cas14a effector protein, a Cas14b effector protein, a Cas14c effector protein, etc.), a Type VI CRISPR-Cas effector protein, etc.) is configured to bind to a protospacer-adjacent motif (PAM) sequence 118 of the input sequence, which is located proximal to the target 110 of the sequence segment 106 (e.g., 5′ or 3′ of the target 110, etc.).

In one embodiment, the CRISPR effector protein 116 is Cas12a (Cpf1), a type V (class II) effector protein, and which binds the PAM sequence (TTTN) 118. The TTTN PAM sequence 118 is expected to occur (in this example) randomly once every about one hundred or so bases in the input sequence. If the gRNA matches the sequence downstream of the PAM sequence 118 (at the target site 110), the CRISPR effector protein 116 may cleave within the input sequence around 17-23 nucleotides downstream of the PAM sequence 118, and produce a 4-5nt staggered cut (e.g. sticky ends, etc.). After cleavage, structures of the target organism containing the sequence segment 106 then aid in the repair of the damage by one of multiple pathways including, for example: non-homologous end joining (NHEJ), homology directed repair (HDR), or microhomology mediated end joining (MMEJ). Non-homologous end joining often results in insertions or deletions at the cut site due to misrepair. Homology directed repair is used to repair the break when a template strand of genetic material is available. And, in microhomology mediated end joining, small regions of microhomology in the surrounding sequence mediate the repair. With that said, the above editing technology is provided as an example only and without limitation of the present disclosure with regard to editing sequences of a genome.

The appropriate genome editing technology may readily be employed by an ordinarily skilled artisan in connection with the editor 102, in accordance with the type and/or degree of editing of the genome required/desired, the CRISPR effector protein used, the size of the experiment (e.g., number of organisms edited, resources committed, etc.), or more generally, the target organism (e.g., plant, animal, etc.) selected and/or required.

With continued reference to FIG. 1, due to differences in characteristics of the sequence segment 106, for example, (and due to many more variables) at the given target 110, the combination of the gRNA 108 and the CRISPR effector protein 116 complexes do not necessarily edit with the same efficiency at/for each sequence segment. Without a way to predict which gRNA 108 and CRISPR effector protein 116 complexes have high or low efficacy, the, researcher (or breeder or other user) has no way to predict the number of samples to treat with the gRNA 108 and CRISPR effector protein 116 complexes in order to recover a desired edit. This uncertainty often results in wasted time and/or resources when executing the experiments(s), under the circumstances that the researcher (or breeder or other user) either does not test enough and fails to achieve the desired edit, or executes too many samples/experiments due to high gRNA efficacy. The ability to predict efficacy of gRNAs, as described herein, thus allows the researcher (or breeder or other user) to scale the experiments to achieve the desired results with the appropriate amount of invested resources. In this manner, for gRNAs predicted to have low efficiency, the researcher, breeder or other user is permitted to either increase the number of samples (e.g., number of cells, number of plant embryos, number of calluses, etc.) exposed to the gRNA 108 and CRISPR effector protein 116 complexes (i.e., to increase a number of samples), or deliver multiple gRNA 108 and CRISPR effector protein 116 complexes to the same individual sample (e.g., multiplexing gRNAs, utilizing high expressing promoters, etc.), or deliver multiple different individual or sets of gRNA 108 and CRISPR effector protein 116 complexes to non-overlapping sets of samples (e.g., increase a number of cells, a number of plant embryos, a number of calluses, etc.) to increase the likelihood of acceptable efficacy, or deliver different sets of gRNA 108 and CRISPR effector protein 116 complexes to different groups of samples to improve and/or increase a number of gRNAs tested.

Thus, the scoring as described in the present disclosure may permit estimation of the number of resources needed in order to obtain the editing of a particular input sequence of a given target organism (e.g., sequence segment 106, etc.).

In connection therewith, the database 104 of the system 100 includes a scoring data structure associated with, or accounting for, differences in one or more of guide nucleic acid characteristics, CRISPR effector protein characteristics, sequence segment characteristics, and target organism. With regard to guide nucleic acid characteristics, it should be appreciated that, in general, a guide nucleic acid is represented by the sequences of nucleotide base: adenine (A), thymine (T)/uracil (U), guanine (G), and cytosine (C). Consequently, a guide nucleic acid (e.g., gRNA) sequence may be characterized by the GC content of the given sequence, or the inclusion of certain combinations of bases, or other characteristics, etc. For example, the scoring data structure may include a score for different characteristics of a guide nucleic acid (e.g., of a gRNA sequence, etc.) based on the presence or absence of the characteristic in the given gRNA (e.g., GC content, Ts, TTs, 5′ T, 5′ G, 5′ C, 5′A, etc.). In some embodiments, the scoring data structure may further include a score for the target organism to be edited (for example, for corn, soy, cotton, canola, brassica, rice, tomato, wheat, etc.).

In some embodiments, the scoring data structure includes scores for one or more characteristics of the sequence segment 106 (e.g., TTTC PAM, TTTG PAM, chromatin accessibility, nucleosome occupancy, histone occupancy, DNA modifications, histone modifications, TA(N)8TA motifs, relative sequence conservation, etc.). For example, chromatin accessibility has been shown to impact editing rates with closed chromatin being associated with lower levels of editing (Strohkendl et al, Sci Adv. 2021 Mar. 10; 7(11):eabd6030. doi: 10.1126/sciadv.abd6030. PMID: 33692102). Features associated with open (accessible) or closed (inaccessible) chromatin could be integrated into the scoring data structure to determine the edit score. There are many ways to assess chromatin accessibility, such as ATAC-Seq (Assay of Transposase accessible chromatin sequencing), Histone ChIp-Seq (chromatin immunoprecipitation sequencing) and bisulfite sequencing to measure methylation (Buenrostro et al., Nat. Methods, 2013 Dec. 10(12):1213-1218); Ricci et al., Methods Mol. Biol. 2020, 2072:101-117). Since all of these assays for chromatin accessibility can be tissue or developmental stage dependent, data from the tissue where the edit is expected to occur would be the most desirable, although other tissues and timepoints may prove valuable. ATAC-Seq data can be also used to assess nucleosome occupancy. In some embodiments, target sites located in regions of open chromatin and low nucleosome occupation, confer a positive addition to their cognate guide nucleic acid's score and target sites in closed chromatin regions and/or high nucleosome occupation can be scored to confer a penalty to their cognate guide nucleic acid's score.

Histone ChIP-seq for H3K4me and H3K27me3 methylated histones indicates active or repressive chromatin marks, respectively. Even though both indicate nucleosome occupancy, active chromatin marks, such as those observed in gene bodies, undergo frequent remodeling. This would allow greater access to the target site by the editing system and correspond to higher editing rates. Conversely, repressive chromatin marks would indicate areas that undergo less remodeling and lower editing rates. H3K27me3, in particular, often displays tissue-specific patterns of distribution, indicating the desirability of generating data from the tissue where the edit is to occur. In some embodiments, the scoring data structure may incorporate data generated from Histone CHIP seq analysis of the sequence segment. In some embodiments, target sites present in H3K4me-enriched regions would confer a positive addition to their cognate guide nucleic acid score while target sites present in H3K27me3-regions could confer a penalty to their score.

In some embodiments, sequence segments may be screened for the presence of TA(N)8TA motifs associated with nucleosome binding. This motif can be found in tandem arrays. When the motif is expanded with multiple repeats, the (N8)TA is repeated (TA(N8)TA(N8)TA for 2 repeats). Both largest number of consecutive repeats on either strand of the dsDNA target and overall sum of the largest number of consecutive repeats from both strands can be measured for a sequence segment comprising the target site about 50 or more nucleotides upstream or downstream of the target site. Not wishing to be bound by a particular theory, higher number of consecutive repeats and sum of consecutive repeats may correspond to lower average editing rates. In some embodiments, the scoring data structure may include scores reflecting a penalty for the presence of repeated TA(N)8TA motifs.

In some embodiments, sequence segments may comprise CG, CHH and/or CHG DNA methylation. CG, CHH and CHG DNA methylation can be monitored, for example, by bisulfite sequencing and are associated with transcriptionally inactive DNA. In some embodiments, the scoring data structure may include scores reflecting DNA methylation of the sequence segment. In some embodiments, sequence segment with high methylation may confer a penalty and/or sequence segment with low methylation may confer an increased score to the cognate guide nucleic acid.

In some embodiments, the scoring data structure may include scores reflecting the presence of small interfering RNA (siRNA), e.g., 24 nt siRNA. The presence of siRNA directed to a sequence is associated with silencing through DNA methylation. In some embodiments, the scoring data structure may include scores reflecting the prevalence of siRNAs targeting the sequence segment. In some embodiments, sequence segment with large numbers of small RNA/siRNA (in terms of diversity or overall abundance) may confer a score reflecting a penalty.

In some embodiments, a researcher, breeder or other user requests to edit a target sequence in multiple different germplasms that in turn may show variations in sequence composition. In this case, the editor computing device identifies available guide nucleic acids with a sufficient match to one or more input sequences from one or more reference genomes and then scores the available guide nucleic acids taking into account conservation of the target sequence in other genomes corresponding to those germplasms in which an edit is desired. Selection of a guide nucleic acid based on an input sequence from a single reference genome runs the risk that the target sequence is not sufficiently conserved in other genomes of interest to allow efficient targeting by the guide nucleic acid and thus efficient editing by the CRISPR editing system. For example, when 76 corn target sequences were analyzed across 10 corn reference genomes, on average, a reference genome had exact matches to about 80% of those 76 gRNA target sequences. Only 40% of the 76 gRNA target sequences were an exact match in all 10 reference genomes. Thus, in one embodiment, the editor computing device incorporates steps for comparison of input sequences from two or more genomes and selects guide nucleic acids predicted to guide efficient editing in different germplasms. In some embodiments, the breeder, researcher or user defines input sequences for different germplasms and specific edit and/or target sequences in the input sequences and the editor computing device identifies each available guide nucleic acid for the input sequences from each germplasm, and provides scores for the available guide nucleic acids based, for example, on the based on its target sequence conservation across two or more germplasms. In some embodiments, the scoring data structure may include a score based on conservation of the target sequence bound by the guide nucleic acid. In some embodiments, the editor computing device can be configured to incorporate a conservation score in the scoring data structure as one of the characteristics scored to determine the edit score of a guide nucleic acid. In some embodiments, the editor computing device may output a conservation score as an independent score to be utilized by the breeder, researcher or other user in conjunction with an edit score.

In some embodiments, based on an effectivity rate defined by the researcher, breeder or other user, the editor computing device may also, optionally, select one or more guide nucleic acid(s) based on the scores and/or a number of samples/experiments to be executed in order to satisfy the request by the researcher, breeder or other user for the edit(s) to the input sequence(s). In this manner, an objective measure of the estimated effectivity rates for different identified guide nucleic acids, for different organisms, different germplasms and edits, etc., is provided, whereby a researcher, breeder or other user (or the editor computing device) may select appropriate guide nucleic acids (at the location, as based on score(s), etc.) to provide sufficient confidence in the output sequence and/or to conserve resources associated with the editing of the input sequence, etc.

Table 1 includes an example of such a scoring data structure, which may be included in the database 104. In Table 1, the example scoring data structure includes different characteristics of a guide nucleic acid (e.g., of a gRNA sequence, etc.) and sequence segment, and also includes a score (for example, for corn and soy (as the given organism)) based on the presence or absence of the characteristic in the given gRNA (e.g., GC content, Ts, TTs, 5′ T, 5′ G, 5′ C, 5′A, etc.) and sequence segment (e.g., TTTC PAM, TTTG PAM, etc.). Table 1 also includes a category or range for characteristics associated with such a category or range, and a score associated with the range of the characteristic. Further in Table 1, as shown, the scores are distinct for different plants, or more broadly, organisms (e.g., corn and soy in Table 1, etc.). For instance, for a gRNA sequence characteristic of GC content, where the category (or range) is less than 30 (<30), the score for corn is −1 and the score for soy is −1. for a gRNA sequence characteristic of GC content, where the category (or range) is between 50 and 60, the score for corn is 1 and the score for soy is 1. Or, for a gRNA sequence characteristic of G at position 6, the score for corn is −0.25 while the score for soy is 0. It should be appreciated that the example scoring data structure in Table 1 is merely an example, and that other scoring data structures (with other data) may be included in other system embodiments. Additionally, other tables including the same or different characteristics and/or associated scores may be included in other system embodiments (e.g., as part of database 104 or otherwise, etc.).

TABLE 1

| | |
|---|---|
| gRNA or Sequence | Score per Plant |

| Segment Characteristic | Category (or Range) | Corn | Soy |
|---|---|---|---|
| GC content | <30 | −1 | −1 |
| GC content | between 30 and 40 | 0.25 | 0.25 |
| GC content | between 40 and 50 | 0.5 | 0.5 |
| GC content | between 50 and 60 | 1 | 1 |
| GC content | between 60 and 70 | 1.25 | 1.25 |
| GC content | >70 | −1 | −1 |
| Ts | >6 | −0.25 | −0.25 |
| TTTC PAM | | −0.5 | −0.5 |
| TTTG PAM | | 0 | −0.25 |
| TTs | >2 | −1 | −1 |
| TTs | 0 | 0.25 | 0.25 |
| 5′ T | | −1 | −1 |
| 5′ G | | 1 | 1 |
| 5′ C | | −0.25 | −0.25 |
| 5′ A | | −0.5 | −0.5 |
| A at position 6 | | 0.25 | 0 |
| G at position 6 | | −0.25 | 0 |
| C at position 6 | | −0.25 | −0.25 |
| C at 23 | | 0.25 | 0.25 |
| A at 23 | | −0.25 | −0.25 |
| G at 23 | | −0.25 | −0.25 |
| A6 +C23 | | 0.75 | 0.5 |
| #Gs 19-22 | >=3 | −0.5 | −0.5 |
| #Gs 19-22 | >0 | −0.25 | −0.25 |

In some embodiments, the scoring data structure may include other characteristics, and/or associated scores may be included (e.g., as part of database 104 or otherwise, etc.). Specifically, for example, the scoring data structure may include additional organisms such as, for example, canola or cotton, etc. Additionally, for example, the scoring data structure may include indexed libraries of multiple reference genomes from the same species of an organism (e.g., genome sequences from multiple different germplasms); SNP (Single nucleotide Polymorphism) library datasets, high-density genome-wide haplotype maps, etc. What's more, the scoring data structure is the product of empirical analysis of the determined effectivity rates of the gRNA(s) for the specific organisms, whereby the scoring data structure may be compiled based on various different sets of gRNAs and/or sequence segments, through one or more techniques suitable to correlate the known effectivity rates to scores associated with characteristics of the specific gRNAs and/or sequence segments, etc. As such, the scoring data structure may be trained based on effectivity rates of the gRNA(s) for the specific organisms, and retrained as additional or different gRNAs are identified and/or built to promote accuracy of the scoring data structure, etc. Consequently, it should be appreciated that the scores in the example scoring data structure (and in the database 104 more generally) may further be trained, and retrained as needed, through one or more machine learning techniques, etc.

In some embodiments, the scores for different gRNAs are then used to assess identified gRNAs relative to one another. Consistent with the above, scores in the scoring data structure for individual characteristics may be determined by leveraging experimental data indicative of the effectiveness of gRNAs based on one or more of the characteristics included in the database 104, as exemplified by the scoring data structure in Table 1. For example, gRNAs were tested to determine an effectivity rate. And, in connection therewith, the different, individual characteristics (alone and/or in combination) are identified as more often associated with relatively high or low efficacy gRNAs. Those characteristics associated with high efficacy gRNAs were set to positive values, whereas those characteristics associated with low efficacy gRNAs were set to negative values. In this manner, the scores included in the scoring data structure are trained based on determined effectivity rates, separately for the target organism (e.g., corn or soy), and then retrained as needed. Scores for one or more characteristics for other organisms could be trained, and retrained, in a similar manner.

Consistent with the example scoring data structure of Table 1, then, the editor 102 is programmed or configured to identify the characteristics from the database 104 (e.g., Table 1, etc.) in the gRNAs and/or sequence segment (e.g., search for each characteristic in the scoring data structure (or other suitable data structure) for the gRNA, etc.) and then aggregate characteristic scores into an edit score for the given gRNA, as described in more detail below. In connection therewith, the editor 102 may be programmed or configured to sum the characteristic scores into the edit score for the given gRNA. Alternatively, the editor 102 may be programmed or configured to average the characteristic scores in order to establish the edit score for the given gRNA. Further, in other embodiments, the editor 102 may be programmed or configured to combine and/or process the characteristic scores in other manners to generate the edit score.

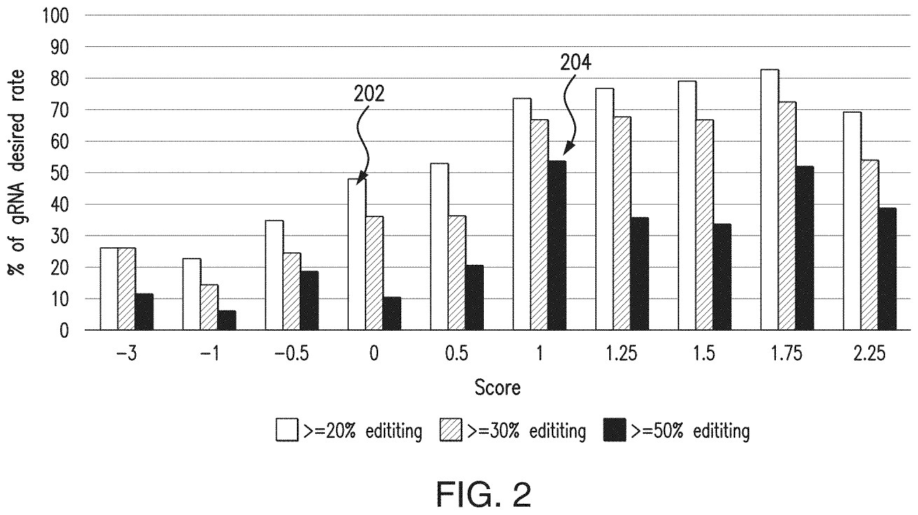

FIG. 2 illustrates an example graphical representation (or chart) of edit scores for multiple different gRNAs evaluated by the editor 102, for the different effectivity rates of greater than or equal to 20% (≥20%), greater than or equal to 30% (≥30%), and greater than or equal to 50% (≥50%). The graphical representation shown in FIG. 2 is based on data derived through experimental editing using the associated gRNAs, with reference to the different efficacy rates. As such, each bar in the chart represents an edit score range and also an effectivity rate for a gRNA. For example, bar 202 is associated with a greater than or equal to 20% rate of effectivity (for achieving a desired edit) and a gRNA having an edit score of greater than or equal to 0 (≥0) and less than 0.5 (<0.5), while bar 204 is associated with a greater than or equal to 50% rate of effectivity and a gRNA having an edit score of greater than or equal to 1.0 (≥1.0) and less than 1.25 (<1.25). The size (e.g., the height, etc.) of the bars then indicate a percentage of gRNAs that have a corresponding edit score and that satisfy the given effectivity rate (as defined by the user and/or the desired edit).

It should be appreciated from the graphic representation in FIG. 2 that a gRNA with an edit score of 1 or higher has higher percentage chance of success for each of the different effectivity rates.

Table 2 below includes the number of gRNAs a researcher, breeder or other user would have to test to find one meeting the given effectivity level based on the experimental data shown in FIG. 2. The numbers in Table 2 represent the numbers of gRNAs in the denoted score ranges, which are needed to satisfy the designated effectivity rate, e.g., greater than or equal to 20% (≥20%), greater than or equal to 30% (≥30%), and greater than or equal to 50% (≥50%), and the average number of effective edits for the gRNAs in the denoted score ranges. As such, for example, Table 2 indicates that eight gRNAs (7.9) should be tested to achieve one gRNA with the desired efficacy rate of a greater than or equal to 50% (≥50%), when the gRNA has an edit score of less than zero. The average effectivity rate is the average effectivity rate for all gRNAs in the score range indicated. As such, for an experiment with 100 samples (e.g., as the number of cells, number of plant embryos, number of calluses, etc.) treated with a gRNA having scores of greater than or equal to one, on average, the effectivity will be about 44 samples edited as desired.

TABLE 2

| gRNA | ≥20% | ≥30% | ≥50% | Average effectivity |
|---|---|---|---|---|
| score | editing | editing | editing | rate |
| <0 | 3.5 | 4.6 | 7.9 | 17.2 |
| >=0 to <1 | 2.0 | 2.8 | 6.6 | 27.2 |
| >=1 | 1.3 | 1.5 | 2.3 | 44.1 |

With reference again to the system 100 in FIG. 1, in use, a researcher, breeder or other user identifies an organism to be edited (e.g., corn, soybean, cotton, canola, wheat, rice, tomato, pepper, etc.) (a target organism having the sequence segment 106 in FIG. 1), and further determines one or more edits to be made to a specific sequence in the organism (e.g., at a single gene or multiple genes, etc.) (e.g., sequence segment 106, etc.). The organism may include, without limitation, a corn plant (broadly, maize plant), a cotton plant, a canola plant, a soybean plant, a barley plant, a rye plant, a rice plant, a tomato plant, a wheat plant, an alfalfa plant, a sorghum plant, an Arabidopsis plant, a cucumber plant, a potato plant, a sweet potato plant, a pepper plant, a carrot plant, an apple plant, a banana plant, a pineapple plant, a blueberry plant, a blackberry plant, a raspberry plant, a strawberry plant, a cucurbit plant, a brassica plant, a citrus plant, a lettuce plant, an onion plant, a pennycress plant, etc. What's more, in some example embodiments, instead of a plant, the organism may be an animal, for example, a cow, a pig, a chicken, etc. Further, in some example embodiments, the organism may include a bacteria, fungi, etc. As explained above, the scoring data structure would be retrained and/or determined for the various characteristics, such as (but not limited to) those in Table 1, based on data relevant to the identified organism. And, the edit(s) to be made to the organism may include, without limitation, one or more deletions, insertions, translocations, deaminations, inversions, gene activation/repression, and epigenetic modifications, etc.

In addition to the organism and the specific edit, the researcher, breeder or other user may identify a desired effectivity rate as a parameter of the edit, such as, for example, greater than or equal to 20% (≥20%), greater than or equal to 30% (≥30%), greater than or equal to 50% (≥50%), etc., although in practice another suitable effectivity range could be chosen based on empirical data from the organism. For example, the researcher, breeder or other user may identify an effectivity rate of at least 20% for a simple edit (e.g., a deletion, etc.), while the researcher, breeder or other user may identify an effectivity rate of at least 50% for a more complex edit (e.g., a site directed insertion, etc.). The effectivity rate selected by the researcher, breeder or other user may be based on analysis of the effectivity rates of various guide nucleic acids (e.g., gRNAs), used alone or in combination in experiments, potentially relative to experimental parameters (e.g., number of edited plants, etc.), etc. These details may vary from organism to organism, and will be defined by the researcher, breeder or other user as needed (e.g., ascertained empirically, etc.).

Then in the system 100, the researcher, breeder or other user defines each of the CRISPR technology(ies) to be used for editing (e.g., which recognizes one or more PAM sequences (e.g., PAM sequence 118, etc.), etc.), the sequence segment 106 of the target organism (in general, or based on proximity), and the effectivity rate to (or for) the editor 102.

In response, the editor 102 is programmed or configured to identify the possible guide nucleic acids (e.g., gRNAs) for the target site(s) in the input sequence 106. For example, where the input sequence 106 includes twelve PAM sequences, the editor 102 may be programmed or configured to identify 12 target sites (e.g., depending on the organism, etc.), and each of the associated guide nucleic acids (e.g., gRNAs) for the different target sites.

The editor 102 is programmed or configured to then score each of the identified gRNAs for the edit. In particular, in this example embodiment, for each of the gRNAs, the editor 102 is programmed or configured to identify each characteristic of the guide nucleic acids (e.g., gRNA) and/or sequence segment as defined in the scoring data structure of the database 104 (e.g., the scoring data structure of Table 1, etc.) and associated score(s). The editor 102 is programmed or configured to then calculate the edit score for the guide nucleic acids (e.g., gRNA), by aggregating each of the one or more scores associated with each identified characteristic in the guide nucleic acids (e.g., gRNA) and/or sequence segment. For example, based on the example scoring data structure of Table 1, where a gRNA for a corn plant includes TTTC, a GC content of 45, A at 23, and 5′G, the editor 102 is programmed or configured to aggregate the characteristic scores of the gRNA into an edit score of 0.75 (i.e., (−0.5)+(0.5)+(−0.25)+(1)=0.75). That said, again, it should be appreciated that different scoring data structures may be used in other embodiments, for corn, soybean or other specific plants/organisms or associated guide nucleic acids (e.g., gRNAs).

In this example embodiment, the editor 102 is then programmed or configured to compile and output a report of identified guide nucleic acids (e.g., gRNAs) and their respective scores to the researcher, breeder or other user (e.g., display the report to the researcher, breeder or other user at the editor computing device 102, or at another computing device, or email or print the report, etc.). In addition, the editor 102 may optionally be programmed or configured to output one or more uniqueness characteristics of the guide nucleic acid target site, with or without the PAM sequence as well as potential off-targets, and other suitable data related to and/or about the identified guide nucleic acids (e.g., which the researcher, breeder or other user may consider helpful in selecting a guide nucleic acid, determining number of samples, etc.). Furthermore, the editor 102 may, optionally, be programmed to output a guide nucleic acid conservation score (indicative of sequence conservation across selected germplasms) when there are a plurality of sequence segments 106 from a plurality of germplasms. In other example embodiments, the editor 102 may be programmed or configured to store the identified guide nucleic acids (e.g., gRNAs) and their respective scores in memory, for example, for subsequent access by the researcher, breeder or other user.

Thereafter, the researcher, breeder or other user, upon receipt of the report (or more generally, the scores of the identified guide nucleic acids (e.g., gRNAs)), selects the desired guide nucleic acid (e.g., gRNA) based on the scores(s). For example, the researcher, breeder or other user may select the guide nucleic acid most likely to produce the desired edit. Additionally, or alternatively, based on the score(s) of the guide nucleic acid and type of edit, the researcher, breeder or other user may then determine the number of guide nucleic acids to be tested based on the edit score of the available guide nucleic acids, the desired effectivity rate and also Table 2 (in this example embodiment) (or similar data structures generated for the desired organism). Additionally, or alternatively, when only guide nucleic acids having a low edit score are available, then the researcher, breeder or other user may select to increase the sample size, number of guide nucleic acids, or number of experiments to compensate for the expected low editing rate (as indicated by the scoring). For example, if the only available guide nucleic acid has a score of 0, the average effectivity rate (as shown in Table 2) is about 17%. To get 100 edited organisms, for example, a sample size of about 590 would be selected by the researcher, breeder or other user (i.e., 590×0.17=100.3). In comparison, a gRNA with a score of greater than or equal to 1 (≥1) is associated with about 44% average effectivity rate, whereby a sample size of only about 230 would be selected (i.e., 230×0.44=101.2). Additional variables, such as, for example, fertility of the organism, viability of the transformed organism, and copy number of transgene insertion may further be included as additional metrics in the report, to inform the user's selection of one or more guide nucleic acids and/or a suitable numbers of samples (whereby each may be determined empirically per organism and/or transformation), etc.

While the user is employed above to select the guide nucleic acids (e.g., gRNA(s)), and also the sample size, number of guide nucleic acids or number of experiments, it should be appreciated, again, that the editor 102 may be programmed or configured in some example embodiments to automatically select the guide nucleic acids based on the score(s) of the respective guide nucleic acids and further to automatically determine a number of samples included in the experiment for the selected guide nucleic acids and Table 2 above (or other suitable table for the specific organism) and to automatically select the sample size for an experiment given the guide nucleic acid and the data above in various other system embodiments. It may also suggest multiple guide nucleic acids to be used either singly in different experiments or multiplexed in one experiment.

Thereafter in the system 100, based on selection of the guide nucleic acid(s) (e.g., gRNA(s)) and the sample sizes of the experiment(s), the experiment(s) is/are executed. In particular, the selected guide nucleic acid(s) (e.g., gRNA(s)) may be synthesized or a nucleic acid sequence encoding the guide nucleic acid (e.g., gRNA) may be cloned into a vector (e.g., a plasmid, etc.), and then transcribing the guide nucleic acid (e.g., gRNA) in vitro, in vivo, in planta, etc. The nucleic acid sequence encoding the CRISPR effector protein(s) may also be cloned into the same vector(s) as the guide nucleic acids or a separate vector, transcribed into mRNA(s) in vitro, in vivo, in planta, or exist in protein form. That said, to impose one or more edits on the sequence segment 106, the guide nucleic acid (e.g., gRNA) 108 and CRISPR effector protein 116, for example, may be delivered (e.g. by mammalian expression vector, Lentiviral transduction, AAV transduction, RNA delivery, plasmid delivery, ribonucleoprotein complexes, lipofection, electroporation, Agrobacteria, etc.) into one or more cells of the target organism. One or more vectors encoding one or more guide nucleic acids (e.g., gRNAs) and/or CRISPR effector proteins may be introduced into a cell (e.g., a plant cell) by any method known to those of skill in the art. In some embodiments, a cell is transformed via bacterial-mediated nucleic acid delivery (e.g., via Agrobacteria), viral-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, mechanical and/or biological mechanism that results in the introduction of nucleic acid into the cell, including any combination thereof. In some embodiments, one or more of polynucleotide(s), polypeptide(s), expression cassette(s), and/or vector(s) may be introduced into a plant cell via Agrobacterium transformation. In some embodiments of the present disclosure, transformation of a cell comprises nuclear transformation. In some embodiments, transformation of a cell comprises plastid transformation (e.g., chloroplast transformation). In some embodiments, a recombinant nucleic acid construct of the present disclosure can be introduced into a cell by breeding. In some embodiments, a ribonucleoprotein complex comprising the guide nucleic acid (e.g., gRNA) and CRISPR effector protein may be introduced into a cell (e.g., a plant cell) by any method known to those of skill in the art (e.g., liposome mediated delivery, microinjection, microparticle bombardment, sonication, infiltration, etc.). In some embodiments, transformation comprises simultaneous transformation of one or more cells from multiple plant germplasms. In some embodiments, transformation comprises collective transformation of populations of seed embryo explants while the individual explants of the population are present together within a single container. The explants of the population may be defined as comprising meristematic tissue or embryonic meristem tissue, which contains plant cells that can differentiate or develop to produce multiple plant structures including, but not limited to, stem, roots, leaves, germ line tissue, and seeds. In some embodiments, the guide nucleic acids (e.g., gRNAs) and CRISPR effector proteins maybe collectively introduced into the explants of the population consecutively, simultaneously, or approximately simultaneously. Simultaneous transformation may be followed by deconvolution, screening, and/or selection of individual explants/germplasm(s) containing the edit. After delivery, the edit can be validated by methods known in the art (e.g. by mismatch-cleavage assay, Polymerase Chain Reaction (PCR), restriction digest, gel electrophoresis, subcloning, Sanger sequencing; next-generation sequencing, Fragment Length Analysis, etc.).

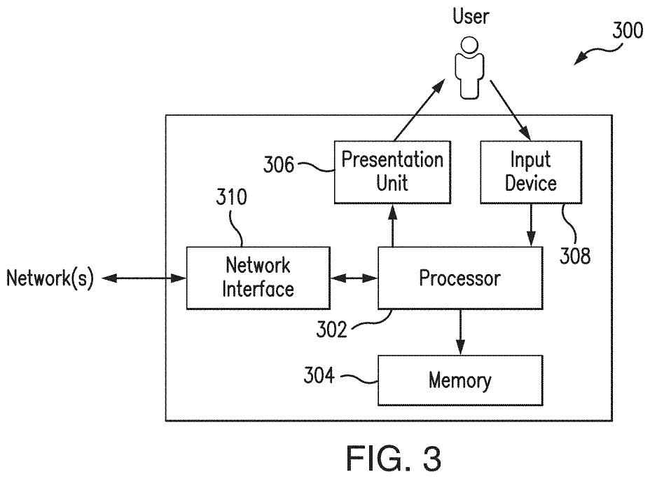

FIG. 3 illustrates an example computing device 300 that can be used in the system 100 of FIG. 1. The computing device 300 may include, for example, one or more servers, workstations, personal computers, laptops, tablets, smartphones, virtual devices, etc. In addition, the computing device 300 may include a single computing device, or it may include multiple computing devices located in close proximity or distributed over a geographic region, so long as the computing devices are specifically configured to operate as described herein. In the example embodiment of FIG. 1, the editor 102 includes and/or is implemented in one or more computing devices consistent with computing device 300 (such that the editor 102 may be considered an editor computing device or genome editor computing device as also referenced herein). In addition, the database 104 may be understood to include and/or be implemented in one or more computing devices, at least partially consistent with the computing device 300. However, the system 100 should not be considered to be limited to the computing device 300, as described below, as different computing devices and/or arrangements of computing devices may be used. In addition, different components and/or arrangements of components may be used in other computing devices.

As shown in FIG. 3, the example computing device 300 includes a processor 302 and a memory 304 coupled to (and in communication with) the processor 302. The processor 302 may include one or more processing units (e.g., in a multi-core configuration, etc.). For example, the processor 302 may include, without limitation, a central processing unit (CPU), a microcontroller, a reduced instruction set computer (RISC) processor, a graphics processing unit (GPU), an application specific integrated circuit (ASIC), a programmable logic device (PLD), a gate array, and/or any other circuit or processor capable of the functions described herein.

The memory 304, as described herein, is one or more devices that permit data, instructions, etc., to be stored therein and retrieved therefrom. In connection therewith, the memory 304 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media for storing such data, instructions, etc. In particular herein, the memory 304 is configured to store data including, without limitation, genome sequences, guide nucleic acid profiles (e.g., identifiers, edit descriptions, sequences, etc.), scoring data structures, effectivity rate data structures, and/or other types of data (and/or data structures) suitable for use as described herein. Furthermore, in various embodiments, computer-executable instructions may be stored in the memory 304 for execution by the processor 302 to cause the processor 302 to perform one or more of the operations described herein (e.g., one or more of the operations of method 400, etc.) in connection with the various different parts of the system 100, such that the memory 304 is a physical, tangible, and non-transitory computer readable storage media. Such instructions often improve the efficiencies and/or performance of the processor 302 that is performing one or more of the various operations herein, whereby such performance may transform the computing device 300 into a special-purpose computing device. It should be appreciated that the memory 304 may include a variety of different memories, each implemented in connection with one or more of the functions or processes described herein.

In the example embodiment, the computing device 300 also includes a presentation unit 306 that is coupled to (and is in communication with) the processor 302 (however, it should be appreciated that the computing device 300 could include output devices other than the presentation unit 306, etc.). The presentation unit 306 may output information (e.g., identified guide nucleic acids, reports associated therewith, etc.), visually or otherwise, to a user of the computing device 300, such as a researcher, breeder or other person or user associated with selection of a nature of edits, etc. It should be further appreciated that various interfaces (e.g., as defined by network-based applications, websites, etc.) may be displayed at computing device 300, and in particular at presentation unit 306, to display certain information to the user. The presentation unit 306 may include, without limitation, a liquid crystal display (LCD), a light-emitting diode (LED) display, an organic LED (OLED) display, an “electronic ink” display, speakers, etc. In some embodiments, presentation unit 306 may include multiple devices. Additionally or alternatively, the presentation unit 306 may include printing capability, enabling the computing device 300 to print text, images, and the like on paper and/or other similar media.

In addition, the computing device 300 includes an input device 308 that receives inputs from the user (i.e., user inputs) such as, for example, selections of organisms, desired edits thereto, and associated parameters including desired effectivity, etc. The input device 308 may include a single input device or multiple input devices. The input device 308 is coupled to (and is in communication with) the processor 302 and may include, for example, one or more of a keyboard, a pointing device, a touch sensitive panel, or other suitable user input devices. In addition, the input device 308 may include, without limitation, sensors disposed and/or associated with the editor 102 and/or the sequencer 104. It should be appreciated that in at least one embodiment an input device 308 may be integrated and/or included with a presentation unit 306 (e.g., a touchscreen display, etc.).

Further, the illustrated computing device 300 also includes a network interface 310 coupled to (and in communication with) the processor 302 and the memory 304. The network interface 310 may include, without limitation, a wired network adapter, a wireless network adapter, a mobile network adapter, or other device capable of communicating to one or more different networks (e.g., one or more of a local area network (LAN), a wide area network (WAN) (e.g., the Internet, etc.), a mobile network, a virtual network, and/or another suitable public and/or private network capable of supporting wired and/or wireless communication among two or more of the parts illustrated in FIG. 1, etc.), including with other computing devices used as described herein.

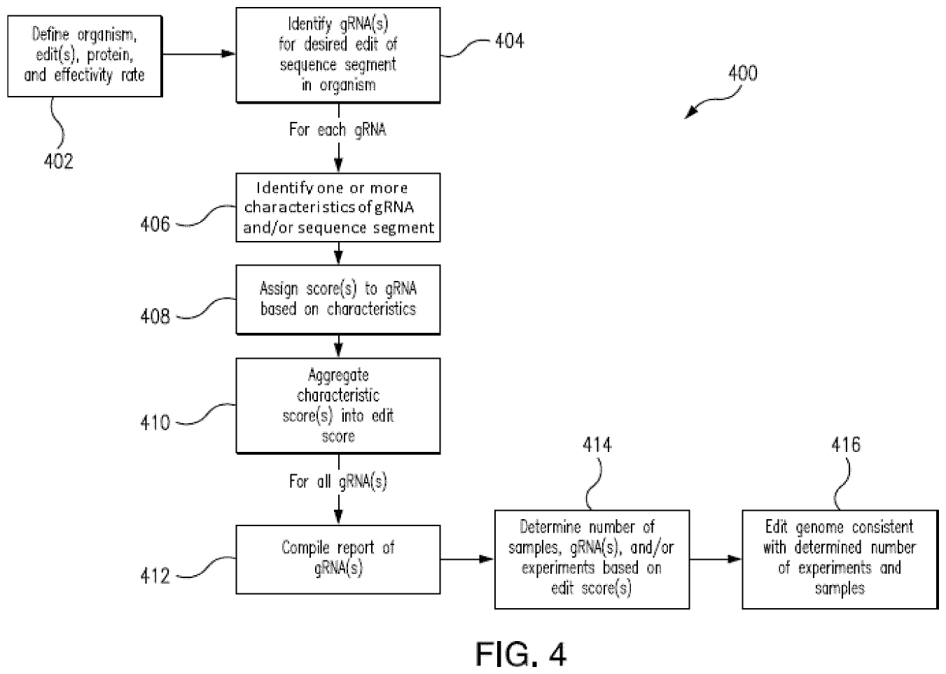

FIG. 4 illustrates an example method 400 for identifying a guide nucleic acid (e.g., a gRNA, etc.) for use in editing an input (or target) genome sequence (of a target organism), based on an effectivity rate of a score associated with the guide nucleic acid. The example method 400 is described herein in connection with the system 100, and may be implemented, in whole or in part, in the editor 102 of the system 100. Further, for purposes of illustration, the example method 400 is also described with reference to the computing device 300 of FIG. 3 (whereby the editor 102 may be considered an editor computing device, etc.). However, it should be appreciated that the method 400, or other methods described herein, are not limited to the system 100 or the computing device 300. And, conversely, the systems, data structures, and the computing devices described herein are not limited to the example method 400.

Initially, at 402, a researcher, breeder or other user defines an organism to be edited, one or more edits for a genome sequence of the organism (or a sequence segment thereof) (e.g., sequence segment 106 in FIG. 1, etc.), the CRISPR effector protein to be used, and an effectivity rate for the edit(s). In some embodiments, a researcher, breeder or other user may want to edit a target sequence present in multiple different germplasms that may in turn vary in sequence composition. In such instances, the researcher, breeder or user may define multiple input sequences from different germplasms. As above, the edit(s) may include one or more of a deletion, insertion, translocation, inversion, substitution, hydrolytic deamination, gene activation/repression, and/or epigenetic modification, and will generally be consistent with one or more desired traits to be advanced in the organism (e.g., where the organism includes a plant product, etc.), or a desired performance in the organism (e.g., a commercial plant or animal product, etc.). The effectivity rate for the edits(s) (e.g., greater than or equal to 20% (≥20%), greater than or equal to 30% (≥30%), and greater than or equal to 50% (≥50%), etc.) may be defined, for example, based on the edit(s), or more specifically, the complexity associated with the edit(s), the organism, etc. In connection therewith, the researcher, breeder or other user, for example, may define a higher effectivity rate for individual guide nucleic acids to make more complex edit(s), etc. Generally, for example (and as used for reference in the method 400), the researcher, breeder or other user, in total, may define an organism as corn, a deletion at a specific location, and a desired effectivity rate for the edit of greater than or equal to 20%, etc.

Next in the method 400, with the desired parameters set by the researcher, breeder or other user, the editor 102 identifies, at 404, the associated guide nucleic acid (e.g., gRNA) for the input sequence in the defined (or target) organism, where the edit is desired or is located, for the selected CRISPR effector protein (e.g., CRISPR effector protein 116, etc.) that will make the edit into the organism. In this specific example, the editor 102 identifies, from the input sequence, each PAM sequence (e.g., PAM sequence 118, etc.) and target location 110 for the given CRISPR effector protein, one or more guide nucleic acids (e.g., gRNAs) for each of the target sequences proximal to PAM sequences. The guide nucleic acids (e.g., gRNAs) may, optionally, be filtered or selected or eliminated based on data such as uniqueness of the target site in the defined organism. In other embodiments, when multiple germplasms are desired to be edited, the guide nucleic acids (e.g., gRNAs) may be filtered based on whether its target site is present in all the germplasms, a majority of the germplasms, some germplasms and/or with what frequency there are mismatches of that target site and/or the evolutionary conservation of the target sequence across multiple germplasms.

For each of the selected or identified guide nucleic acids (e.g., gRNAs), the editor 102 identifies, at 406, one or more characteristics of the guide nucleic acid sequence (e.g., for gRNA 108 in FIG. 1, etc.) and/or sequence segment. In this example embodiment, the editor 102 identifies whether the guide nucleic acid (e.g., gRNA) 108 includes, for example, any of the characteristics included in the example scoring data structure of Table 1 (as included in the database 104). In particular, the editor 102 identifies a characteristic included in the scoring data structure (e.g., in database 104, etc.) and then searches in the guide nucleic acid sequence and/or the input sequence surrounding the target site for that characteristic. When the characteristic is found/identified, the editor 102 may determine it is either present or not, or count a number of occurrences of the characteristic in the guide nucleic acid sequence, the input sequence surrounding the target site, etc. The editor 102 further repeats the search for each of the characteristics in the scoring data structure (or other suitable data structure), As such, for example, the editor 102 may search for occurrences of T or TT or GC content, etc., characteristics in the guide nucleic acid (e.g., gRNA) 108, one or more characteristics of the sequence segment (e.g., TTTC PAM, TTTG PAM, chromatin accessibility, nucleosome occupancy, histone occupancy, DNA modifications, histone modifications, TA(N)8TA motifs, relative sequence conservation, etc.) and/or matches the characteristics of the guide nucleic acid (e.g., gRNA) 108 to the characteristics of Table 1.

In turn, based on the identified characteristics of the given guide nucleic acid (e.g., gRNA) 108 and/or the input sequence surrounding the target site 110, the editor 102 assigns, at 408, one or more scores to the guide nucleic acid (e.g., gRNA) 108, where (in this example) each of the assigned scores is associated with the identified characteristic in Table 1. These scores, which may be referred to as characteristic scores, are then aggregated, by the editor 102, at 410, into an edit score for the guide nucleic acid (e.g., gRNA) 108. For instance, as described in the system 100, based on the example scoring data structure of Table 1, where a guide nucleic acid (e.g., gRNA) for a corn plant includes a GC content of 45, A at 23, and 5′G, and the input sequence surrounding the target site 110 includes TTC, the editor 102 is programmed or configured to aggregate the characteristic scores of the guide nucleic acid (e.g., gRNA) into an edit score of 0.75 (i.e., (−0.5)+(0.5)+(−0.25)+(1)=0.75). In the example method 400, the editor 102 then repeats steps 406-410 for each of the selected or identified guide nucleic acids (e.g., gRNAs) and aggregates an edit score for each of the selected guide nucleic acids (e.g., gRNAs) in this manner.

When multiple germplasms are desired to be edited, the editor 102 may further assign, at 408, a conservation score as one of the characteristics scored for each guide nucleic acid based on the sequence conservation of the target sequence across each of the input sequences from different germplasm. The methods by which to achieve the assessment of target site conservation are numerous. For example, a breeder, researcher or other user may incorporate two or more input sequences from indexed libraries of reference genomes into the database 104. A spacer sequence 112 of a given guide nucleic acid sequence 108 is then matched to the target sites contained in input sequences from different reference genomes by genome editor 102 to produce an output that indicates via a conservation score whether the guide nucleic acid sequence is an exact match to the target site. In some embodiments, a conservation score may take into consideration PAM flexibility of the CRISPR editing system with perfect guide nucleic acid and target site match. In some embodiments, guide nucleic acid sequences matching a target site with a PAM corresponding to the CRISPR editing system may receive an increased score and/or guide nucleic acid sequences matching a target site with an imperfect PAM may receive a score penalty. In some embodiments, the editor can also identify potential off-targets or imperfect matches of the reference genome to a selected guide nucleic acid. In some embodiments, guide nucleic acid sequences with no or a low number of potential off-targets may receive an increased score and/or guide nucleic acid sequences with one or more off-targets may receive a score penalty. In some embodiments, the score penalty for a given guide nucleic acid may increase in relation to the number of potential off-targets.

In some embodiments, a conservation score may be calculated when the breeder, researcher or other user queries a set of target sequences 110 using the genomic positions of those sequences to a database of SNPs, where SNP information can be overlaid onto the coordinates of the target sequence using a common set of location index (mapping every SNP and target sequence to a reference sequence of given coordinates). In some embodiments, guide nucleic acids corresponding to target sequences mapping to genomic positions with conserved SNPs among two or more reference genomes may receive an increased conservation score and/or guide nucleic acid sequences corresponding to target sequences mapping to genomic positions without conserved SNPs may receive a conservation score penalty. In some embodiments, a set of input sequences from reference genomes that represent the diversity of all haplotypes in a breeding program are provided. In some embodiments, the breeder, researcher or other user may then select lines of interest to edit and use pedigree relationships to determine which reference genome represents the haplotype or sequence at the target site and then query for guide nucleic acids in that reference genome only. In some embodiments, the breeder, researcher or other user may query for guide nucleic acids with high conservation scores in multiple reference genomes.

In some embodiments, a researcher, breeder, or other user specifies one or more guide nucleic acids corresponding to a selected target in a specified reference genome, for e.g. B73 maize germplasm. The genome editor 102 may be programmed to query the specified guide nucleic acid(s) (with coordinates relative to the reference genome) against a library of SNPs, indels or variants that represent many types of germplasm. The positions of these variants are relative (or can be calculated to concord with) to the specified reference genome. The genome editor 102 then provides the researcher, breeder or other user with a report that describes which nucleotide positions in the specified guide nucleic acid(s) have variants in the queried genomes. In this way, pre-calculated or highly confident variants can be rapidly assessed relative to genome coordinates of the specified guide nucleic acid(s).

In some embodiments, an editor computing device 102 can be configured to incorporate the conservation score as one of the characteristic scores used to calculate the edit score of the given gRNA at 410. In other embodiments, the editor computing device 102 may output the conservation score as an independent score in the report at 412 which can be utilized as a part of an index of weighted features in conjunction with the edit score. For example, the breeder, researcher, user, or the editor computing device may combine the weights of a guide nucleic acid conservation score with a guide nucleic acid edit score to identify an optimal guide nucleic acid or set of guide nucleic acids that have likelihood of guiding editing in one or more selected germplasms. In some embodiments, guide nucleic acids that are conserved in certain types of germplasm such as specific gender classes in hybrid crop production (male vs female), certain relative maturities, certain germplasm associated to certain geographies (i.e. South America or tropical germplasm), or having other desirable germplasm traits are selected.

With continued reference to FIG. 4, once edit scores and optionally conservation scores are generated for each of the identified guide nucleic acids (e.g., gRNAs), the editor 102 can optionally store the identified guide nucleic acid (e.g., gRNA) sequences and their respective scores in memory (e.g., memory 304, etc.), for example, for subsequent access, use, etc. Additionally, in this example, the editor 102 compiles, at 412, a report including, without limitation, the identified guide nucleic acids (e.g., gRNAs), locations of the target sites, uniqueness of the target site in the organism, number of predicted off-target sites, location of predicted off-target sites, conservation of the target site and/or the edit scores for each of the selected or identified guide nucleic acids (e.g., gRNAs), etc. The report may include such data for all of the identified guide nucleic acids (e.g., gRNAs) together. Or, a separate report may be generated containing such data for each identified guide nucleic acid (e.g., gRNA).And, with the report, at 414, the editor 102 (or, potentially, the researcher, breeder or other user) determines the sample size, number of guide nucleic acids (e.g., gRNAs), and/or number of experiments to be performed, based on the data structure, for example, the data structure of Table 2, and the edit scores (optionally the conservation scores) for the different guide nucleic acids (e.g., gRNAs) (alone or in combination), in order to achieve the identified effectivity rate defined at step 402.

In particular, at 414, based on the edit scores, for example, where only guide nucleic acids (e.g., gRNAs)associated with an edit score of 0.5 are available, the editor 102 (or, potentially, the researcher, breeder or other user) may determine that at least seven guide nucleic acids (e.g., gRNAs) (i.e., 6.6) should be tested to find one guide nucleic acid with a greater than or equal to 50% effectivity rate, as shown in Table 2. Alternatively, since the average editing rate of a guide nucleic acid with a score of greater than 0 but less than 1.0 is about 27%, the editor 102 (or, potentially, the researcher, breeder or other user) may determine to double the sample size to achieve the same number of edited individuals. In this example embodiment, the sample size is associated with a simple edit with a single guide nucleic acid (e.g., gRNA).

The editor 102, or associated user, then, at 416, causes the genome to be edited consistent with the determined number of samples and/or experiments.

In a further implementation of the method 400, when two or more guide nucleic acids (e.g., gRNAs) are used (or needed) for complex edits such as inversions, deletions between two target sequences, transversions, translocations, insertions etc., success of obtaining the desired editing outcome may be understood to be dependent upon the lowest effectivity rate from among the two or more guide nucleic acids (e.g., gRNAs), as shown in Table 3 below. Table 3 illustrates a percent of experiments with greater than or equal to 5% (≥5%) deletion rate between two guide nucleic acids (e.g., gRNAs) compared to the activity of individual guide nucleic acids (e.g., gRNAs) in the pair (where each pair include a minimum (relatively lower) and a maximum (relatively higher) editing rate fell within the effectivity range in the guide nucleic acid (e.g., gRNA) rate column.

TABLE 3

| Guide nucleic acid | | |
|---|---|---|
| (e.g., gRNA) rate | Minimum editing rate | Maximum editing rate |
| 100-50 | 100.0 | 60.9 |
| 49-30 | 100.0 | 50.0 |
| 29-20 | 25.0 | 20.0 |
| 19-0 | 20.0 | 25.0 |

In this example implementation, the desired edit was a deletion between the two target sequences and the desired effectivity rate of achieving that edit was 5%. Effectivity rates of each guide nucleic acid and the desired deletion between the target sites were determined for different pairs of guide nucleic acids (e.g., gRNAs). The percent of experiments achieving the 5% deletion between the two target sites was examined in light of the effectivity rate of the selected guide nucleic acids. Overall experimental success of the deletion between two target sites was determined to correlate more closely with the minimum editing rate (as identified in Table 3). Based on the guide nucleic acids (e.g., gRNAs) available, along with the relative editing rates, then, the editor 102 (or potentially, the researcher, breeder or other user) may proceed to determine the number of guide nucleic acids to test, the number of experiments and/or a sample size for achieving the desired edit.

For deletion between two target sites with guide nucleic acids at 5% effectivity, for example, Table 3 shows that a minimum effectivity rating of the guide nucleic acids (e.g., gRNAs) is 30% was sufficient to obtain the 5% effectivity rating in 100% of experiments conducted. If the desired deletion has multiple guide nucleic acids (e.g., gRNAs) with a score of greater than or equal to 1 (≥1) at both ends, the editor 102 (or potentially, the researcher, breeder or other user) multiplexes the 1-2 guide nucleic acids (e.g., gRNAs) at each end of the deletion because, based on Table 2, for example, the editor 102 (or potentially, the researcher, breeder or other user) determines to test 1.5 guide nucleic acids (e.g., gRNAs) to find one guide nucleic acid (e.g., gRNA) that has a 30% effectivity rating. If all identified guide nucleic acids (e.g., gRNAs) have a score of less than 0 (<0), five guide nucleic acids (e.g., gRNAs) (i.e., 4.6 from Table 2) would need to be tested on each end to find a pair with greater than 30% (≥30%) efficacy. That said, if only one set of guide nucleic acids (e.g., gRNAs) each with a score of less than zero were available in this example, the average effectivity rate of the minimum guide nucleic acid (e.g., gRNA) is about 17% (as shown in Table 2). In this permutation of the example, the editor 102 (or potentially the researcher, breeder or other user) determines, from Table 3 (e.g., for a guide nucleic acid (e.g., gRNA) rate between 19-0, etc.), that only one in five guide nucleic acid pairs (e.g., minimum editing rate of 20%, etc.) are expected to give the desired 5% deletion between the target sites. The editor 102 (or potentially, the researcher, breeder or other user) is then able to compensate for lower rates by determining to increase the sample size (e.g., number of cells, number of plant embryos, number of calluses, etc.) of the experiment with the specific guide nucleic acids (e.g., gRNAs).

In this manner, the editor 102 (or potentially, the researcher, breeder or other user) is permitted to determine the number of experiments and/or sample for the defined edit based on the scores determined at step 410 and reported at step 412 for the complex edit. The editor 102, or associated user, then again at step 416, causes the genome to be edited consistent with the determined number of guide nucleic acids (e.g., gRNAs), samples and/or experiments.

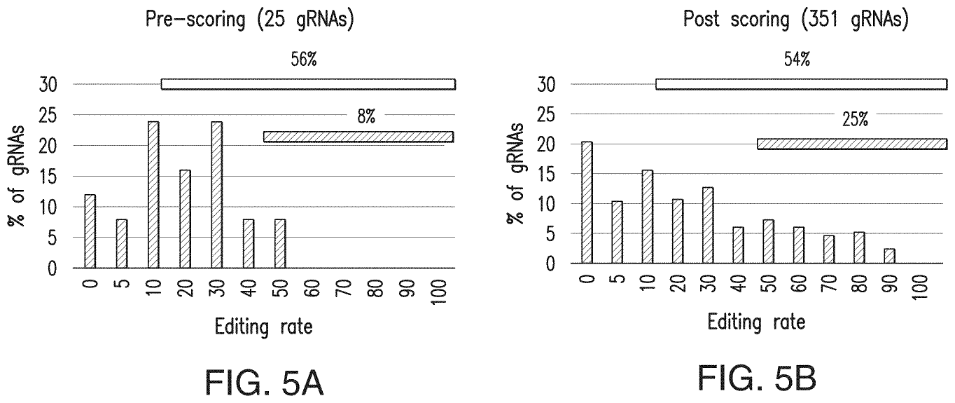

The scoring, by the editor 102, described in the method 400 provides substantial insight into the usability of the different guide nucleic acids (e.g., gRNAs). In particular, higher effectivity rates are often more desirable in achieving a desired editing outcome (e.g., due to reduction in resources, etc.). For example, FIG. 5A illustrates pre-scoring effectivity rates of certain gRNAs, and FIG. 5B illustrates post-scoring effectivity rates of the gRNAs. As shown, there is about a four-times increase in the proportion of gRNAs with an effectivity rate of greater than or equal to 50% (≥50%), e.g., post-scoring, as compared to selecting gRNAs in the absence of the scoring described above, e.g., pre-scoring. It should be noted that in FIG. 5B, twenty-five percent of gRNAs are at or above 50%, while it is only 8% in FIG. 5A. In addition, it should be noted that some of the gRNAs in FIG. 5B are further available as higher scoring gRNAs (e.g., 70%, 80% and 90% percent effectivity rates), where none were available in the pre-scoring of FIG. 5A.

In another example implementation of method 400, the researcher, breeder or other user may define, at 402, an edit as a frame shift in the exon 2 of the Bmr3 gene, or potentially, delete the entire exon2, and also define a 20% effectivity rate for an individual gRNA in exon 2 and a 30% effectivity rate for guide nucleic acids (e.g., gRNAs) causing the exon deletion. The editor 102 is then relied on to select, at 404, the appropriate gRNAs for LbCas12a and to score, at 406-410, the guide nucleic acids (e.g., gRNAs) as described above. In particular, for each of the selected or identified guide nucleic acids (e.g., gRNAs), the editor 102 identifies, at 406, one or more characteristics of the guide nucleic acids (e.g., gRNAs) and/or target sequence (or sequence segment), for example, relating to use of the selected guide nucleic acids (e.g., gRNAs) in performing the desired edit to the organism. Then, based on the identified characteristics, the editor 102 assigns, at 408, one or more scores to the guide nucleic acids (e.g., gRNAs) 108, where (in this example) each of the assigned scores is associated with the identified characteristic, for example, the characteristics in Table 1. And these scores, which may be referred to as characteristic scores, are aggregated, by the editor 102, at 410, into an edit score for the selected guide nucleic acid (e.g., gRNA).

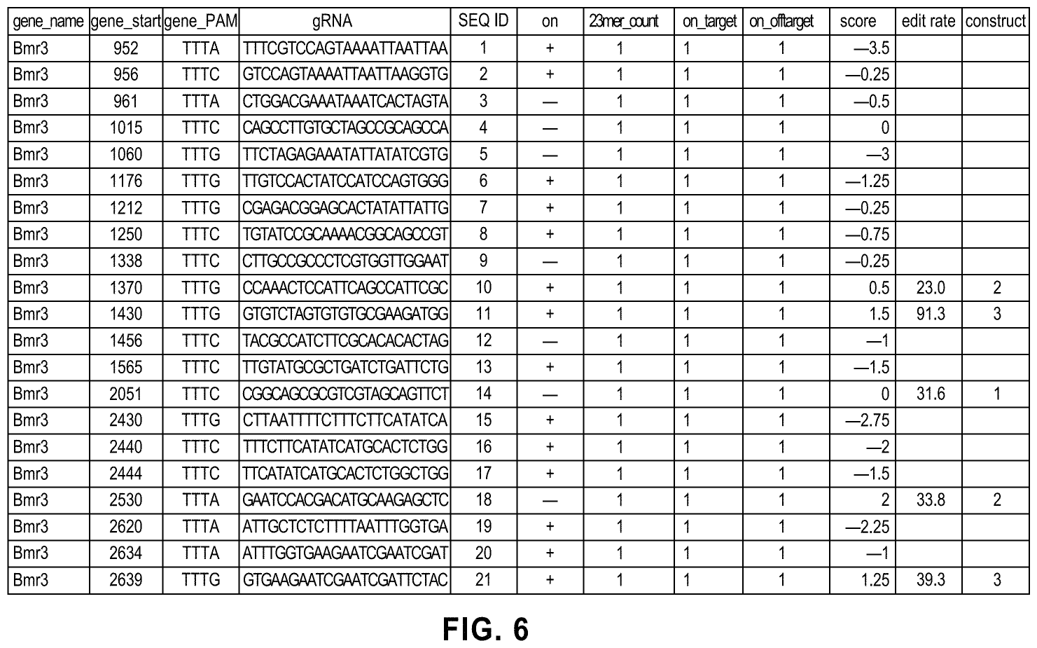

Next, once edit scores are generated for each of the identified guide nucleic acids (e.g., gRNAs), the editor 102 compiles, at 412, a report including, without limitation, the identified gRNAs, locations of the gRNAs, uniqueness in the organism, off-target and scores for each of the selected or identified gRNAs, etc. An example report compiled by the editor 102 is illustrated in FIG. 6. In FIG. 6, it should be appreciated that the gene_name is the gene name and the gene_start is the location of the first base in sequence. The gRNA is the sequence belonging to the target portion (or target sequence) of the gRNA; the gene_PAM is the PAM sequence specific to the CRISPR effector protein; the ori is the orientation relative to the user provided sequence; the 23_mer_count is the number of times the target sequence was identified in the genome; the on_target is the number of times the target sequence has the appropriate on-target PAM sequence (i.e., the total number of on-targets); the on_offtargets is the number of times the target sequence is found within the genome with three or less mismatches and adjacent to the appropriate PAM sequence (a combination of on and off targets); and the score is the score derived from gRNA characteristics as described above. While in this example the target sequence is within the open reading frame of a gene, one of skill in the art would recognize that the target sequence may be anywhere in the genome, for example, in a promoter, an enhancer, 5′ or 3′ untranslated regions, an intron, a non-genic region, etc.

As shown in FIG. 6, as an example (and without limitation), the gRNA CGGCAGCGCGTCGTAGCACTTCT (at gene_start 2051) (SEQ ID NO:14) is in the exon 2. Since this specific gRNA has a score of 0, the score is used, by the editor 102 or the researcher, breeder or other user, to predict the gRNA as having about a 50% chance of editing at a 20% effectivity rate for the request from the researcher, breeder or other user, as defined in FIG. 2 (at 202). Consequently, an alternate edit (e.g., deleting the entire exon 2, etc.) is also considered. For deleting the entire exon 2, in FIG. 6, there are two gRNAs having scores greater than or equal to 1 (≥1) downstream of the exon and one gRNA having a score greater than or equal to 1 (≥1) upstream of the exon. For deletion between 2 gRNAs, a minimum editing rate of 30% was desired, as determined by the editor 102 or the researcher, breeder or other user from Table 3, and about 67% of gRNAs with a score of greater than or equal to 1 (≥1) would reach this threshold, as determined from FIG. 2.

Further in this example implementation, in order to enhance the chance of success, three constructs were designed and transformed, one with the exon specific gRNA and two constructs to delete exon 2 as denoted by the construct column in FIG. 6. Construct 2 had a non-recommended gRNA with a score 0.5 because the researcher, breeder or other user, in this example implementation, decided to create the exon 2 deletion for validation and the selected gRNA was the highest scoring gRNA 5′ of the exon after the gRNA at 1430.

In connection with the above, the constructs were executed and/or transformed, and then the organisms assayed for editing activity using standard sequencing methods known in the art (as instructed by the edit rate column FIG. 6). As a result, Construct 1 created edits in exon 2 in 31.6% of the organisms, while Construct 3 had a greater than 30% (>30%) editing at each gRNA with greater than 5% (>5%) of the organisms having the desired exon 2 deletion. Both Construct 1 and Construct 3 were at rates high enough to be introduced into a breeding pipeline. Conversely, Construct 2 did not produce any deletions between the two gRNAs. This is consistent with a recommendation to choose gRNAs with a score of greater than or equal to 1 (≥1) to improve experimental success.

In view of the above, the systems and methods herein provide for enhanced sequence editing based on scoring associated with effectivity rates of different guide nucleic acids (e.g., gRNAs). In particular, when faced with the decisions related to editing an input sequence for an organism, the researcher, breeder or other user is often left with trial and error, or over estimation as a tool to select specific guide nucleic acids (e.g., gRNAs), and then, the experiment sizes and number of samples. The lack of objective metrics for the selection of guide nucleic acids (e.g., gRNAs) result in the selection of less efficient guide nucleic acids (e.g., gRNAs), and the selection of experiment and/or number of samples in excess of what is needed, or probable to result in the desire edits. The systems and methods herein provide an object measure of the guide nucleic acid (e.g., gRNA), based on empirical data as represented in scoring data structures. The scoring data structures are trained and/or retrained based on suitable data for the specific organism and thereby serve to inform the breeder, researcher, or other user of the specific effectivity rate of the guide nucleic acid (e.g., gRNA), and the corresponding experimental scale and/or number of samples to achieve the desired edit in the organism.

Consequently, then, researchers, breeders, and other users are more likely to select the appropriate level of resources, for selected guide nucleic acids (e.g., gRNA(s)), thereby reducing wasted resources associated with too many or too few samples and/or experiments or wasted time when an experiment fails to yield the desired edit and must be redone.

An editing system useful to modify a genome (e.g., a sequence or sequence segment thereof, etc.), based on guide nucleic acid(s) (e.g., gRNA(s)) identified as described herein, can be any CRISPR editing system now known or later developed, which system can modify genetic material of the genome in a target specific manner.

For example, a CRISPR-Cas editing system can include, but is not limited to, a Type I CRISPR-Cas system, a Type II CRISPR-Cas system (e.g., a Cas9 system), a Type III CRISPR-Cas system, a Type IV CRISPR-Cas system, a Type V CRISPR-Cas system (e.g., a Cas12a (Cpf1) system, a Cas12b system, a Cas12c (C2c3) system, a Cas12d (CasY) system, a Cas12e (CasX) system, a Cas12g system, a Cas12h system, a Cas12i system, a C2c1 system, a C2c4 system, a C2c5 system, a C2c8 system, a C2c9 system, a C2c10 system, a Cas14a system, a Cas14b system, a Cas14c system, etc.), a Type VI CRISPR-Cas system (non-limiting examples of CRISPR effector proteins include Cas1, Cas1B, Cas2, Cas3, Cas4, Cas5, Cas6, Cas7, Cas8, Cas9 (also known as Csn1 and Csx12), Cas10, Cas 12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, Mad7). In some embodiments, the CRISPR-Cas editing system can comprise one or more sequence-specific nucleic acid binding domains (DNA/RNA binding domains) that can be from, for example, a Cas9, a Cas12a, a Cas12b, a Cas12c (C2c3), a Cas12d (CasY), a Cas12e (CasX), a Cas12g, a Cas12h, a Cas12i, a C2c1, a C2c4, a C2c5, a C2c8, a C2c9, a C2c10, a Cas14a, a Cas14b, a Cas14c, etc., and an effector domain that modifies the nucleic acid. Examples of effector domains include cleavage domains, a deaminase (e.g., a cytosine deaminase, an adenine deaminase, etc.), a Uracil DNA glycosylase inhibitor, a reverse transcriptase, a Dna2 polypeptide, and/or a 5′ flap endonuclease (FEN), etc. In some embodiments, an editing system can comprise one or more polynucleotides, including, but is not limited to, an extended guide nucleic acid, and/or a reverse transcriptase template.

In some embodiments, a method of modifying a genome sequence (e.g., a sequence segment thereof, etc.) as described herein may comprise contacting the targeted nucleic acid with a base-editing fusion protein (e.g., a CRISPR-Cas9 effector protein or domain, a CRISPR-Cas12a effector protein or domain, etc.) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing, for example, the identified genetic segment. In some embodiments, the nuclease activity of the CRISPR effector protein (e.g., a CRISPR-Cas9 effector protein or domain, a CRISPR-Cas12a effector protein or domain, etc.) has been inactivated. In some embodiments, a base editing fusion protein and guide nucleic acid may be encoded by one or more expression cassettes. In some embodiments, the genome of the target organism may be contacted with a base editing fusion protein and an expression cassette encoding a guide nucleic acid(s). In some embodiments, the base editing fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, modifying a genome sequence (e.g., a sequence segment thereof, etc.) as described herein may comprise contacting a target nucleic acid with a CRISPR-Cas system (e.g., a Type I CRISPR-Cas system, a Type II CRISPR-Cas system (e.g., a Cas9 system), a Type III CRISPR-Cas system, a Type IV CRISPR-Cas system, a Type V CRISPR-Cas system (e.g., a Cas12a (Cpf1) system, a Cas12b system, a Cas12c (C2c3) system, a Cas12d (CasY) system, a Cas12e (CasX) system, a Cas12g system, a Cas12h system, a Cas12i system, a C2c1 system, a C2c4 system, a C2c5 system, a C2c8 system, a C2c9 system, a C2c10 system, a Cas14a system, a Cas14b system, a Cas14c system, etc.), a Type VI CRISPR-Cas system, etc.), that induces cleavage of one or both strands of the target nucleic acid thereby modifying, for example, the target sequence. In some embodiments, a CRISPR-Cas system may be comprised in one or more expression cassettes. In some embodiments, the sequence-specific editing system may be provided as ribonucleoproteins (RNPs). In some embodiments, the target nucleic acid may be contacted with a CRISPR-Cas system comprising a guide nucleic acid that comprises a sequence which is at least partly complementary to the target nucleic acid. In some embodiments, a cell may be contacted with more than one CRISPR-Cas system that may target one or more target nucleic acids in the cell. In some embodiments, a cell may be contacted with one or more CRISPR-Cas systems (e.g., a CRISPR-Cas9 editing system and/or a CRISPR-Cas12a editing system, etc.) comprising one or more guide nucleic acids that may target one or more target nucleic acids in the cell.

In some embodiments, a CRISPR-Cas system may modify a genetic segment of a genome sequence by inducing a single-strand break (a “nick”). In some embodiments, a CRISPR-Cas system may modify a genetic segment by inducing a double-strand break. In some embodiments, the double-strand break can be blunt. In some embodiments, the double-strand break can be staggered, which produces “sticky ends”. In some embodiments, a CRISPR-Cas system may comprise a cytidine deaminase. In an aspect, a “modification” comprises the hydrolytic deamination of cytidine or deoxycytidine to uridine or deoxyuridine, respectively. In some embodiments, a CRISPR-Cas system may comprise an adenine deaminase. In an aspect, a “modification” comprises the hydrolytic deamination of adenine or adenosine. In an aspect, a “modification” comprises the hydrolytic deamination of adenosine or deoxyadenosine to inosine or deoxyinosine, respectively. In an aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C”, “G” or “T” in a nucleic acid sequence. In some embodiments, a “modification” comprises the substitution of an “C” for a “A”, “G” or “T” in a nucleic acid sequence. In some embodiments, a “modification” comprises the substitution of an “G” for a “A”, “C” or “T” in a nucleic acid sequence. In some embodiments, a “modification” comprises the substitution of an “T” for a “A”, “C” or “G” in a nucleic acid sequence. In some embodiments, a “modification” comprises the substitution of an “C” for a “U” in a nucleic acid sequence. In some embodiments, a “modification” comprises the substitution of an “G” for a “A” in a nucleic acid sequence. In some embodiments, a “modification” comprises the substitution of an “A” for a “G” in a nucleic acid sequence. In some embodiments, a “modification” comprises the substitution of an “T” for a “C” in a nucleic acid sequence. In some embodiments, a “modification” comprises the insertion of one or more transgenes. In some embodiments, a “modification” comprises the exchange of segments between homologous chromosomes. In some embodiments, a “modification” comprises the exchange of segments between heterologous chromosomes.

In view of the above, the systems and methods permit guide nucleic acid (e.g., gRNA) sequences to be scored in a manner predictive of the effectiveness of the guide nucleic acids (e.g., gRNAs) to achieve specific edits, whether simple or complex. Consequently, the systems and methods herein may then identify guide nucleic acids (e.g., gRNAs) having different effectivity rates (e.g., relatively high effectivity rates, for example, above about 50%, etc.), which permits the editor to provide for scaling of experiments appropriately and to, therefore, provide efficiencies and/or savings in time and resources.

With that said, it should be appreciated that the functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques, including computer software, firmware, hardware or any combination or subset thereof, wherein the technical effect may be achieved by performing at least one of the following operations: (a) for each of multiple guide nucleic acid sequences, for a desired edit of a sequence segment of a target organism: (i) identifying one or more characteristics of the guide nucleic acid and/or sequence segment; (ii) assigning based on a scoring data structure, a score to the guide nucleic acid sequence for each of the identified one or more characteristics; and (iii) aggregating the assigned scores into an edit score for the guide nucleic acid sequence; (b) compiling a report, wherein the report includes the multiple guide nucleic acid sequences and the edit score for each of the multiple guide nucleic acid sequences, thereby permitting selection, from the report, of at least one of the multiple guide nucleic acid sequences based on the associated edit score; (c) receiving a request for the desired edit; (d) identifying the multiple guide nucleic acid sequences based on the desired edit of the sequence segment of the target organism and/or a location in the sequence segment; (e) identifying the at least one of the multiple guide nucleic acid sequences based on the associated edit score; and (f) determining, by the genome editor computing device, a number of experiments and/or samples for the at least one of the multiple guide nucleic acid sequences, based on the edit score and the defined effectivity rate, and/or the effectivity rate of the at least one of the multiple guide nucleic acid sequences.

As will also be appreciated based on the foregoing specification, the above-described embodiments of the disclosure may be implemented using computer programming or engineering techniques, including computer software, firmware, hardware or any combination or subset thereof, wherein the technical effect may be achieved by performing at least one of the following operations: (a) for each of multiple guide nucleic acid (e.g., gRNA) sequences, for a desired edit of a sequence segment of a target organism: (i) identifying, by a genome editor computing device, one or more characteristics of the guide nucleic acid and/or sequence segment; (ii) assigning, by the genome editor computing device, based on a scoring data structure, a score to the guide nucleic acid sequence for each of the identified one or more characteristics; and (iii) aggregating, by the genome editor computing device, the assigned scores into an edit score for the guide nucleic acid sequence; (b) determining, by the genome editor computing device, for at least one of the multiple guide nucleic acid sequences, a number of experiments and/or samples to achieve the desired edit of the sequence segment of the target organism, based on the edit score for the at least one of the multiple guide nucleic acid sequences; (c) receiving a request for the desired edit; (d) identifying the multiple guide nucleic acid sequences based on the desired edit of the sequence segment of the target organism and/or a location in the sequence segment; (e) compiling, by the genome editor computing device, a report, wherein the report includes the multiple guide nucleic acid sequences and the edit score for each of the multiple guide nucleic acid sequences; and (f) identifying the at least one of the multiple guide nucleic acid sequences from the report, based on the associated edit score for the at least one of the multiple guide nucleic acid sequences.

Examples and embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may also be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

### SPECIFIC EMBODIMENTS

The following embodiments are provided by way of illustration and are not intended to be limiting of the invention, unless specified.

A first embodiment relates to a computer-implemented method for use in identifying one or more mechanisms for editing a genome sequence, the method comprising:

for each of multiple guide nucleic acid sequences, for a desired edit of a sequence segment of a target organism: identifying, by a genome editor computing device, one or more characteristics of the guide nucleic acid sequence and/or sequence segment; assigning, by the genome editor computing device, based on a scoring data structure, a score to the guide nucleic acid sequence for each of the identified one or more characteristics; and aggregating, by the genome editor computing device, the assigned scores into an edit score for the guide nucleic acid sequence; and then compiling, by the genome editor computing device, a report, wherein the report includes the multiple guide nucleic acid sequences and the edit score for each of the multiple guide nucleic acid sequences, thereby permitting selection, from the report, of at least one of the multiple guide nucleic acid sequences based on the associated edit score.

A second embodiment relates to the computer-implemented method of embodiment 1, further comprising: receiving a request for the desired edit; and identifying the multiple guide nucleic acid sequences based on the desired edit of the sequence segment of the target organism and/or a location in the sequence segment.

A third embodiment relates to the computer-implemented method of embodiment 2, wherein the request includes a defined effectivity rate for the desired edit of the sequence segment.

A fourth embodiment relates to the computer-implemented method of any one of embodiments 1-3, wherein identifying one or more characteristics of the guide nucleic acid sequence includes identifying the one or more characteristics, based on a scoring data structure, in the guide nucleic acid sequence.

A fifth embodiment relates to the computer-implemented method of any one of embodiments 1-3, wherein identifying one or more characteristics includes identifying the one or more characteristics, based on a scoring data structure, in the sequence segment.

A sixth embodiment relates to the computer-implemented method of any one of embodiments 1-5, wherein the one or more characteristics are independently selected from: GC content; a defined combination of adenine, thymine, guanine, and cytosine; a position of one or more of adenine, thymine, guanine, and cytosine; and a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

A seventh embodiment relates to the computer-implemented method of any one of embodiments 1-6, wherein aggregating the assigned scores into the edit score for the guide nucleic acid sequence includes summing the scores assigned to the guide nucleic acid sequence for each of the identified one or more characteristics.

An eight embodiment relates to the computer-implemented method of any one of embodiments 1-7, further comprising: identifying the at least one of the multiple guide nucleic acid sequences, from the report, based on the associated edit score; and/or determining, by the genome editor computing device, a number of experiments and/or samples for the at least one of the multiple guide nucleic acid sequences, based on the edit score and a defined effectivity rate from the request and/or based on an effectivity rate of the at least one of the multiple guide nucleic acid sequences.

A ninth embodiment relates to the computer-implemented method of any one of embodiments 1-8, further comprising editing the sequence segment of the target organism using the selected at least one of the multiple guide nucleic acid sequences.

A tenth embodiment relates to the computer-implemented method of any one of embodiments 1-9, wherein the target organism includes a plant.

An eleventh embodiment relates to the computer-implemented method of embodiment 10, wherein the plant includes either a corn plant or a soy plant.

A twelfth embodiment relates to the computer-implemented method of any one of embodiments 1-9, wherein the target organism includes an animal.

A thirteenth embodiment relates to the computer-implemented method of any one of embodiments 1-12, wherein the guide nucleic acid is selected from gRNA, gRNA/DNA, and gDNA.

A fourteenth embodiment relates to a system for use in identifying one or more mechanisms for editing a genome sequence, the system comprising: a genome editor computing device configured to: for each of multiple guide nucleic acid sequences, for a desired edit of a sequence segment of a target organism: identify one or more characteristics of the guide nucleic acid sequence and/or sequence segment; assign, based on a scoring data structure, a score to the guide nucleic acid sequence for each of the identified one or more characteristics; and aggregate the assigned scores into an edit score for the guide nucleic acid sequence; and then store, in memory in communication with the genome editor computing device, the multiple guide nucleic acid sequences and the edit score for each of the multiple guide nucleic acid sequences, thereby permitting selection of at least one of the multiple guide nucleic acid sequences based on the associated edit score.

A fifteenth embodiment relates to the system of embodiment 14, wherein the genome editor computing device is further configured to: receive a request for the desired edit; and identify the multiple guide nucleic acid sequences based on the desired edit of the sequence segment of the target organism and/or a location in the sequence segment.

A sixteenth embodiment relates to the system of embodiment 15, wherein the request includes a defined effectivity rate for the desired edit of the sequence segment.

A seventeenth embodiment relates to the system of any one of embodiments 14-16, wherein the genome editor computing device is configured, in order to identify the one or more characteristics of the guide nucleic acid sequence, to identify the one or more characteristics, based on a scoring data structure, in the guide nucleic acid sequence.

An eighteenth embodiment relates to the system of any one of embodiments 14-17, wherein the genome editor computing device is configured, in order to identify the one or more characteristics, to identify the one or more characteristics, based on a scoring data structure, in the sequence segment.

A nineteenth embodiment relates to the system of any one of embodiments 14-18, wherein the one or more characteristics are selected from: GC content; a defined combination of adenine, thymine, guanine, and cytosine; a position of one or more of adenine, thymine, guanine, and cytosine; and a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

A twentieth embodiment relates to the system of any one of embodiments 14-19, wherein the genome editor computing device is configured, in order to aggregate the assigned scores into the edit score for the guide nucleic acid sequence, to sum the scores assigned to the guide nucleic acid sequence for each of the identified one or more characteristics.

A twenty first embodiment relates to the system of any one of embodiments 14-20, wherein the genome editor computing device is further configured to: identify the at least one of the multiple guide nucleic acid sequences, from the memory, based on the associated edit score; and/or determine a number of experiments and/or samples for the at least one of the multiple guide nucleic acid sequences, based on the edit score and a defined effectivity rate from the request and/or based on an effectivity rate of the at least one of the multiple guide nucleic acid sequences.

A twenty second embodiment relates to the system of any one of embodiments 14-21, wherein the target organism includes a plant.

A twenty third embodiment relates to the system of embodiment 22, wherein the plant includes either a corn plant or a soy plant.

A twenty fourth embodiment relates to the system of any one of embodiments 14-21, wherein the target organism includes an animal.

A twenty fifth embodiment relates to the system of any one of embodiments 14-24, wherein the genome editor computing device is further configured to compile a report including the multiple guide nucleic acid sequences and the edit score for each of the multiple guide nucleic acid sequences.

A twenty sixth embodiment relates to the system of any one of embodiments 14-25, wherein the guide nucleic acid is selected from gRNA, gRNA/DNA, and gDNA.

A twenty seventh embodiment relates to a system for use in identifying one or more mechanisms for editing a genome sequence, the system comprising: at least one genome editor computing device configured to: receive a request for a desired edit of a sequence segment of a target organism; identify multiple guide nucleic acid sequences based on the desired edit of the sequence segment of the target organism and/or based on a location in the sequence segment in the target organism; for each of the identified multiple guide nucleic acid sequences, for the desired edit: identify one or more characteristics of the guide nucleic acid sequence and/or sequence segment; assign, based on a scoring data structure, a score to the guide nucleic acid sequence for each of the identified one or more characteristics; and aggregate the assigned scores into an edit score for the guide nucleic acid sequence; and then compile a report, wherein the report includes the multiple guide nucleic acid sequences and the edit score for each of the multiple guide nucleic acid sequences, thereby permitting selection, from the report, of at least one of the multiple guide nucleic acid sequences based on the associated edit score.

A twenty eighth embodiment relates to the system of embodiment 27, wherein the request includes a defined effectivity rate for the desired edit; and wherein the at least one genome editor computing device is further configured to: identify the at least one of the multiple guide nucleic acid sequences, from the report, based on the associated edit score; and determine a number of experiments and/or samples for the at least one of the multiple guide nucleic acid sequences, based on the edit score and the defined effectivity rate from the request and/or based on an effectivity rate of the at least one of the multiple guide nucleic acid sequences.

A twenty-ninth embodiment relates to the system of embodiment 27 or embodiment 28, wherein the request includes a defined effectivity rate for the desired edit of the sequence segment.

A thirtieth embodiment relates to the system of any one of embodiments 27-29, wherein the at least one genome editor computing device is configured, in order to identify the one or more characteristics of the guide nucleic acid sequence, to identify the one or more characteristics, based on a scoring data structure, in the guide nucleic acid sequence.

A thirty-first embodiment relates to the system of any one of embodiments 27-29, wherein the at least one genome editor computing device is configured, in order to identify the one or more characteristics, to identify the one or more characteristics, based on a scoring data structure, in the sequence segment.

A thirty-second embodiment relates to the system of any one of embodiments 27-31, wherein the one or more characteristics are selected from: GC content; a defined combination of adenine, thymine, guanine, and cytosine; a position of one or more of adenine, thymine, guanine, and cytosine; and a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

A thirty-third embodiment relates to the system of any one of embodiments 27-32, wherein the at least one genome editor computing device is configured, in order to aggregate the assigned scores into the edit score for the guide nucleic acid sequence, to sum the scores assigned to the guide nucleic acid sequence for each of the identified one or more characteristics.

A thirty-fourth embodiment relates to the system of any one of embodiments 27-33, wherein the target organism includes a plant.

A thirty-fifth embodiment relates to the system of embodiment 34, wherein the plant includes either a corn plant or a soy plant.

A thirty-sixth embodiment relates to the system of any one of embodiments 27-33, wherein the target organism includes an animal.

A thirty-seventh embodiment relates to the system of any one of embodiments 27-36, wherein the guide nucleic acid is selected from gRNA, gRNA/DNA, and gDNA.

A thirty-eighth embodiment relates to a computer-implemented method for use in identifying one or more guide nucleic acids for editing a genome sequence, the method comprising: for each of multiple guide nucleic acid sequences, for a desired edit of a sequence segment of a target organism: identifying, by a genome editor computing device, one or more characteristics of the guide nucleic acid sequence and/or sequence segment; assigning, by the genome editor computing device, based on a scoring data structure, a score to the guide nucleic acid sequence for each of the identified one or more characteristics; and aggregating, by the genome editor computing device, the assigned scores into an edit score for the guide nucleic acid sequence; and then determining, by the genome editor computing device, for at least one of the multiple guide nucleic acid sequences, a number of experiments and/or samples to achieve the desired edit of the sequence segment of the target organism, based on the edit score for the at least one of the multiple guide nucleic acid sequences.

A thirty-ninth embodiment relates to the computer-implemented method of embodiment 38, wherein determining the number of experiments and/or samples for the at least one of the multiple guide nucleic acid sequences to achieve the desired edit of the sequence segment of the target organism is further based on an effectivity rate of the at least one of the multiple guide nucleic acid sequences.

A fortieth embodiment relates to the computer-implemented method of embodiment 38, further comprising: receiving a request for the desired edit; and identifying the multiple guide nucleic acid sequences based on the desired edit of the sequence segment of the target organism and/or a location in the sequence segment.

A forty-first embodiment relates to the computer-implemented method of embodiment 40, wherein the request includes a defined effectivity rate for the desired edit of the sequence segment; and wherein determining the number of experiments and/or samples for the at least one of the multiple guide nucleic acid sequences to achieve the desired edit of the sequence segment of the target organism is further based on the defined effectivity rate included in the request.

A forty-second embodiment relates to the computer-implemented method of any one of embodiments 38-41, wherein identifying one or more characteristics of the guide nucleic acid sequence includes identifying the one or more characteristics, based on a scoring data structure, in the guide nucleic acid sequence.

A forty-third embodiment relates to the computer-implemented method of any one of embodiments 38-41, wherein identifying one or more characteristics includes identifying the one or more characteristics, based on a scoring data structure, in the sequence segment.

A forty-fourth embodiment relates to the computer-implemented method of any one of embodiments 38-43, wherein the one or more characteristics are selected from: GC content; a defined combination of adenine, thymine, guanine, and cytosine; a position of one or more of adenine, thymine, guanine, and cytosine; and a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

A forty-fifth embodiment relates to the computer-implemented method of any one of embodiments 38-44, wherein aggregating the assigned scores into the edit score for the guide nucleic acid sequence includes summing the scores assigned to the guide nucleic acid sequence for each of the identified one or more characteristics.

A forty-sixth embodiment relates to the computer-implemented method of any one of embodiments 38-45, further comprising: compiling, by the genome editor computing device, a report, wherein the report includes the multiple guide nucleic acid sequences and the edit score for each of the multiple guide nucleic acid sequences; and identifying the at least one of the multiple guide nucleic acid sequences from the report, based on the associated edit score for the at least one of the multiple guide nucleic acid sequences.

A forty-seventh embodiment relates to the computer-implemented method of any one of embodiments 38-46, further comprising editing the sequence segment of the target organism using the selected at least one of the multiple guide nucleic acid sequences.

A forty-eighth embodiment relates to the computer-implemented method of any one of embodiments 38-47, wherein the target organism includes a plant.

A forty-ninth embodiment relates to the computer-implemented method of embodiment 48, wherein the plant includes either a corn plant or a soy plant.

A fiftieth embodiment relates to the computer-implemented method of any one of embodiments 38-47, wherein the target organism includes an animal.

A fifty-first embodiment relates to the computer-implemented method of any one of embodiments 38-50, wherein the guide nucleic acid is selected from gRNA, gRNA/DNA, and gDNA.

A fifty-second embodiment relates to a system for use in identifying one or more guide nucleic acids for editing a genome sequence, the system comprising: at least one genome editor computing device configured to: for each of multiple guide nucleic acid sequences, for a desired edit of a sequence segment of a target organism: identify one or more characteristics of the guide nucleic acid sequence and/or sequence segment; assign, based on a scoring data structure, a score to the guide nucleic acid sequence for each of the identified one or more characteristics; and aggregate the assigned scores into an edit score for the guide nucleic acid sequence; and then determine, for at least one of the multiple guide nucleic acid sequences, a number of experiments and/or samples to achieve the desired edit of the sequence segment of the target organism, based on the edit score for the at least one of the multiple guide nucleic acid sequences.

A fifty-third embodiment relates to the system of embodiment 52, wherein the at least one genome editor computing device is configured, in order to determine the number of experiments and/or samples for the at least one of the multiple guide nucleic acid sequences to achieve the desired edit of the sequence segment of the target organism, to determine the number of experiments and/or samples further based on an effectivity rate of the at least one of the multiple guide nucleic acid sequences.

A fifty-fourth embodiment relates to the system of embodiment 52, wherein the at least one genome editor computing device is further configured to: receive a request for the desired edit; and identify the multiple guide nucleic acid sequences based on the desired edit of the sequence segment of the target organism and/or a location in the sequence segment.

A fifty-fifth embodiment relates to the system of embodiment 54, wherein the request includes a defined effectivity rate for the desired edit of the sequence segment; and wherein the at least one genome editor computing device is configured, in order to determine the number of experiments and/or samples for the at least one of the multiple guide nucleic acid sequences to achieve the desired edit of the sequence segment of the target organism, to determine the number of experiments and/or samples further based on the defined effectivity rate included in the request.

A fifty-sixth embodiment relates to the system of any one of embodiments 52-55, wherein the at least one genome editor computing device is configured, in order to identify one or more characteristics of the guide nucleic acid sequence, to identify the one or more characteristics in the guide nucleic acid sequence based on a scoring data structure.

A fifty-seventh embodiment relates to the system of any one of embodiments 52-55, wherein the at least one genome editor computing device is configured, in order to identify one or more characteristics, to identify the one or more characteristics in the sequence segment based on a scoring data structure.

A fifty-eighth embodiment relates to the system of any one of embodiments 52-57, wherein the one or more characteristics are selected from: GC content; a defined combination of adenine, thymine, guanine, and cytosine; a position of one or more of adenine, thymine, guanine, and cytosine; and a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

A fifty-ninth embodiment relates to the system of any one of embodiments 52-58, wherein the at least one genome editor computing device is configured, in order to aggregate the assigned scores into the edit score for the guide nucleic acid sequence, to sum the scores assigned to the guide nucleic acid sequence for each of the identified one or more characteristics.

A sixtieth embodiment relates to the system of any one of embodiments 52-59, wherein the at least one genome editor computing device is further configured to: compile a report including the multiple guide nucleic acid sequences and the edit score for each of the multiple guide nucleic acid sequences; and identify the at least one of the multiple guide nucleic acid sequences from the report, based on the associated edit score for the at least one of the multiple guide nucleic acid sequences.

A sixty-first embodiment relates to the system of any one of embodiments 52-60, wherein the at least one genome editor computing device is further configured to store, in memory in communication with the genome editor computing device, the multiple guide nucleic acid sequences and the edit score for each of the multiple guide nucleic acid sequences.

A sixty-second embodiment relates to the system of any one of embodiments 52-61, wherein the target organism includes a plant.

A sixty-third embodiment relates to the system of embodiment 62, wherein the plant includes either a corn plant or a soy plant.

A sixty-fourth embodiment relates to the system of any one of embodiments 52-61, wherein the target organism includes an animal.

A sixty-fifth embodiment relates to the system of any one of embodiments 52-64, wherein the guide nucleic acid is selected from gRNA, gRNA/DNA, and gDNA.

A sixty-sixth embodiment relates to a non-transitory computer-readable storage medium including executable instructions for identifying one or more guide nucleic acids for use in editing a genome sequence, which when executed by at least one processor of a genome editor computing device, cause the at least one processor to perform one or more of the operations recited in embodiments 1, 14, 27, 38, and 52.

A sixty-seventh embodiment relates to a system as described herein and/or as illustrated in FIG. 1.

A sixty-eighth embodiment relates to a method as described herein and/or as illustrated in FIG. 3.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in identifying one or more mechanisms for editing a genome sequence, the method comprising:
for each of multiple guide nucleic acid sequences, for a desired edit of a sequence segment of a target organism:
identifying, by a genome editor computing device, one or more characteristics of the guide nucleic acid sequence and/or sequence segment;
assigning, by the genome editor computing device, based on a scoring data structure, a score to the guide nucleic acid sequence for each of the identified one or more characteristics; and
aggregating, by the genome editor computing device, the assigned scores into an edit score for the guide nucleic acid sequence; and then
compiling, by the genome editor computing device, a report, wherein the report includes the multiple guide nucleic acid sequences and the edit score for each of the multiple guide nucleic acid sequences, thereby permitting selection, from the report, of at least one of the multiple guide nucleic acid sequences based on the associated edit score.

2. The computer-implemented method of claim 1, further comprising:
receiving a request for the desired edit; and
identifying the multiple guide nucleic acid sequences based on the desired edit of the sequence segment of the target organism and/or a location in the sequence segment.

3. The computer-implemented method of claim 2, wherein the request includes a defined effectivity rate for the desired edit of the sequence segment.

4. The computer-implemented method of claim 1, wherein identifying one or more characteristics includes identifying the one or more characteristics, based on a scoring data structure, in the guide nucleic acid sequence.

5. The computer-implemented method of claim 1, wherein identifying one or more characteristics includes identifying the one or more characteristics, based on a scoring data structure, in the sequence segment.

6. The computer-implemented method of claim 1, wherein the one or more characteristics are independently selected from: GC content; a defined combination of adenine, thymine, guanine, and cytosine; a position of one or more of adenine, thymine, guanine, and cytosine; a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

7. The computer-implemented method of claim 1, wherein aggregating the assigned scores into the edit score for the guide nucleic acid sequence includes summing the scores assigned to the guide nucleic acid sequence for each of the identified one or more characteristics.

8. The computer-implemented method of claim 2, further comprising:
identifying the at least one of the multiple guide nucleic acid sequences, from the report, based on the associated edit score; and/or
determining, by the genome editor computing device, a number of experiments and/or samples for the at least one of the multiple guide nucleic acid sequences, based on the edit score and a defined effectivity rate from the request and/or based on an effectivity rate of the at least one of the multiple guide nucleic acid sequences.

9. The computer-implemented method of claim 1, further comprising editing the sequence segment of the target organism using the selected at least one of the multiple guide nucleic acid sequences.

10. The computer-implemented method of claim 1, wherein the target organism includes a plant.

11.-13. (canceled)

14. A system for use in identifying one or more mechanisms for editing a genome sequence, the system comprising:
a genome editor computing device configured to:
for each of multiple guide nucleic acid sequences, for a desired edit of a sequence segment of a target organism:
identify one or more characteristics of the guide nucleic acid sequence and/or sequence segment;
assign, based on a scoring data structure, a score to the guide nucleic acid sequence for each of the identified one or more characteristics; and
aggregate the assigned scores into an edit score for the guide nucleic acid sequence; and then
store, in memory in communication with the genome editor computing device, the multiple guide nucleic acid sequences and the edit score for each of the multiple guide nucleic acid sequences, thereby permitting selection of at least one of the multiple guide nucleic acid sequences based on the associated edit score.

15. The system of claim 14, wherein the genome editor computing device is further configured to:
receive a request for the desired edit; and
identify the multiple guide nucleic acid sequences based on the desired edit of the sequence segment of the target organism and/or a location in the sequence segment.

16. The system of claim 14, wherein the request includes a defined effectivity rate for the desired edit of the sequence segment.

17. The system of claim 14, wherein the genome editor computing device is configured, in order to identify the one or more characteristics, to identify the one or more characteristics, based on a scoring data structure, in the guide nucleic acid sequence.

18. The system of claim 14, wherein the genome editor computing device is configured, in order to identify the one or more characteristics, to identify the one or more characteristics, based on a scoring data structure, in the sequence segment.

19. The system of claim 14, wherein the one or more characteristics are selected from: GC content; a defined combination of adenine, thymine, guanine, and cytosine; a position of one or more of adenine, thymine, guanine, and cytosine; a number of one or more of adenine, thymine, guanine, and cytosine; TTTC PAM; TTTG PAM; chromatin accessibility; nucleosome occupancy; histone occupancy; DNA modifications; histone modifications; TA(N)8TA motifs; and relative target site sequence conservation.

20. The system of claim 14, wherein the genome editor computing device is configured, in order to aggregate the assigned scores into the edit score for the guide nucleic acid sequence, to sum the scores assigned to the guide nucleic acid sequence for each of the identified one or more characteristics.

21. The system of claim 14, wherein the genome editor computing device is further configured to:
identify the at least one of the multiple guide nucleic acid sequences, from the memory, based on the associated edit score; and/or
determine a number of experiments and/or samples for the at least one of the multiple guide nucleic acid sequences, based on the edit score and a defined effectivity rate from the request and/or based on an effectivity rate of the at least one of the multiple guide nucleic acid sequences.

22. The system of claim 14, wherein the target organism includes a plant.

23.-24. (canceled)

25. The system of claim 14, wherein the genome editor computing device is further configured to compile a report including the multiple guide nucleic acid sequences and the edit score for each of the multiple guide nucleic acid sequences.

26.-71. (canceled)
